SEQUENCE LISTING (8 sequences)

SEQ ID NO: 1 - DNA, 2625 bp, synthetic construct
  source 1..2625: mol_type = other DNA
gattcatacaccacctcaacagacgattcgtctaatgacactgccgacagtgtctctgatggtgtgattttacacgcttggtgttggtctttcaacacaatcaagaacaatttgaagcaaattcacgatgcaggttacactgccgttcaaacctcccctgtcaatgaagtcaaagttggtaattctgctagtaagtctttgaacaactggtactggttataccaaccaacaaagtactcgattggtaactattacttaggtaccgaagctgaattcaagtccatgtgtgcagctgccaaggagtacaacatcagaattattgttgatgctaccttgaatgacaccacaagtgactactcagctatttcggatgaaatcaaatccattagtaattggactcatggcaatacacagatatccaactggtcagacagggaggatgtcacccaaaactctctccttggtttgtatgattggaacactcaaaattcccaagtccaaacatacctaaagaactacttggaacgtctaatatcagatggggcaagcggttttcgttacgatgcagccaaacatatcgaattgccatcacaatacgacggttcatatggttccaatttttggccaaatatcactgacaatggtagtgaattccaatatggcgaagttttgcaagattctatttccaaagaatccgattacgctaattacatgtcagtaacagcctctaattatggtaatactattagaaatgccctgaaaaacagagatttcactgctagcacattacaaaatttcaatatttctgtccccgctagcaagttggttacttgggttgaatctcatgacaactatgcaaacgatgaccaagtttctacctggatgaatagttccgatattaaactaggttgggccgtagtggcctcaagatctggaagtgttccattatttttcgacagaccagttgacggtggtaatggtacccgttttcctggatctagcgaaattggtgacgccggttcttcgctttattatgacaaggctgttgtggcggttaacaagttccacaacgccatggctggtcaatctgaatacatttcaaacccaaacggtaacaccaaaatttttgaaaacgaaagaggttctaagggtgtcgttttcgctaatgcttcggatggcagctattctctatctgttaagacatctcttgctgacggtacctacgaaaataaggccggaagtgacgagttcactgttaaaaacggttatttgacaggtactatccaaggtagagaagtagtcgtattatatggcgatccaacttcaagctcgtcctcgtctaccactactgaaactaagaaggtgtattttgaaaaaccatcctcctggggttccacagtctatgcctatgtctacaacaaaaacactaataaggctataaccagcgcatggccaggtaaagagatgactgctttaggtaatgatgagtataaattagacctggatacagatgaagatgattccgacttggcagtaattttcaccgatgggaccaaccaaactcctgcagccaacaaggctgggttcaccttcacagcagacgcgacgtacgatcagaacggtgttgttaagacctctgactcatcttcgtcgtcctccactaccaccgaaacaaaaaaagtgtattttgaaaagccttcatcttgggggtccactgtctacgcctacgtttataataaaaacacgaacaaagctatcaccagtgcttggcccggtaaggaaatgaccgctcttggaaatgacgaatataaattggatttggatactgatgaagatgatagtgatctagctgttatctttactgatggtacaaaccaaacgccggcagctaacaaggcaggtttcacttttaccgctgatgccacttatgatcaaaacggtgtggttaagacatctgacagttcttcatcatcttccagtacaactacggaaactaagaaagtttacttcgaaaagccatcttcgtggggctctacggtttacgcttatgtttataacaagaatacaaataaagcaattacttccgcttggcctggtaaggaaatgactgcgttaggcaacgacgaatacaagttagatttagataccgatgaagatgatagtgatttggctgtgatcttcactgatggaaccaaccagactccagctgctaacaaagcaggctttacctttactgctgatgccacttatgaccagaatggtgttgtcaagacctccgatagctcctcttcctcgtcaactactacagaaacgaagaaggtttactttgagaagccaagtagttggggttctacagtttatgcttacgtatacaataaaaatactaataaagcgatcactagcgcctggccaggtaaagaaatgacagctttgggcaatgacgaatacaaattggaccttgacactgacgaggacgactccgatttggctgttatatttaccgatggtactaatcaaacgcctgctgcaaataaagctggtttcacatttaccgccgatgctacttacgatcagaacggtgtcgtcaaaacatctgattcttcgtccacctcttctacatcataa

SEQ ID NO: 2 - protein, 874 aa, unidentified
  source 1..874: note = Lactobacillus plantarum S21
DSYTTSTDDSSNDTADSVSDGVILHAWCWSFNTIKNNLKQIHDAGYTAVQTSPVNEVKVGNSASKSLNNWYWLYQPTKYSIGNYYLGTEAEFKSMCAAAKEYNIRIIVDATLNDTTSDYSAISDEIKSISNWTHGNTQISNWSDREDVTQNSLLGLYDWNTQNSQVQTYLKNYLERLISDGASGFRYDAAKHIELPSQYDGSYGSNFWPNITDNGSEFQYGEVLQDSISKESDYANYMSVTASNYGNTIRNALKNRDFTASTLQNFNISVPASKLVTWVESHDNYANDDQVSTWMNSSDIKLGWAVVASRSGSVPLFFDRPVDGGNGTRFPGSSEIGDAGSSLYYDKAVVAVNKFHNAMAGQSEYISNPNGNTKIFENERGSKGVVFANASDGSYSLSVKTSLADGTYENKAGSDEFTVKNGYLTGTIQGREVVVLYGDPTSSSSSSTTTETKKVYFEKPSSWGSTVYAYVYNKNTNKAITSAWPGKEMTALGNDEYKLDLDTDEDDSDLAVIFTDGTNQTPAANKAGFTFTADATYDQNGVVKTSDSSSSSSTTTETKKVYFEKPSSWGSTVYAYVYNKNTNKAITSAWPGKEMTALGNDEYKLDLDTDEDDSDLAVIFTDGTNQTPAANKAGFTFTADATYDQNGVVKTSDSSSSSSSTTTETKKVYFEKPSSWGSTVYAYVYNKNTNKAITSAWPGKEMTALGNDEYKLDLDTDEDDSDLAVIFTDGTNQTPAANKAGFTFTADATYDQNGVVKTSDSSSSSSTTTETKKVYFEKPSSWGSTVYAYVYNKNTNKAITSAWPGKEMTALGNDEYKLDLDTDEDDSDLAVIFTDGTNQTPAANKAGFTFTADATYDQNGVVKTSDSSSTSSTS

SEQ ID NO: 3 - DNA, 1470 bp, synthetic construct
  source 1..1470: mol_type = other DNA
aatacaggtcatttccaagcctactctggttacacagttgctcgttccaacttcacccaatggattcacgaacaacctgccgtgtcatggtattatttgcttcagaatattgactacccagaaggccagttcaaatcggccaagcctggtgttgttgtggccagcccatctacttcagagccagattacttttaccaatggactagagatactgcaattactttcttgagtttgattgctgaagttgaagaccattctttttcaaacactactttggctaaggtcgttgaatactacatttcaaatacatacaccttacaaagagtatcgaacccatcaggtaactttgacagcccaaaccatgatggtttaggtgaaccaaagtttaatgtggatgataccgcatatactgcttcttggggtcgtcctcaaaatgacggtccagctttgagagcttatgctatttctaggtatctgaatgccgtcgccaaacacaacaacggtaagttgctgctcgcgggccaaaacggtataccgtattcttctgcctctgatatctactggaaaattattaaacctgatttacaacatgtttccacccattggtctacctccggatttgatttgtgggaagagaaccaaggtactcacttcttcacggcactagtgcagttgaaagctctatcttatggtattcctttgtccaagacttataatgatccagggtttacctcgtggttggaaaagcaaaaggatgctttaaattcctacataaattcttccggtttcgttaattcaggcaaaaagcacattgtcgaatctccacaacttagttctagaggtggtttggactcagctacctatatcgccgctctaatcacccacgatattggtgacgatgacacctacactccattcaatgtcgacaacagctatgtcttaaacagtttatattacttattggttgataacaagaatcgttataaaatcaacggaaactacaaggctggtgctgctgttggtagatatcctgaagatgtttacaatggtgtcggaacttctgaaggtaatccatggcaattggccactgcctacgctggtcaaactttttatacattagcttacaactccttgaagaacaagaaaaatttagtaattgaaaaattgaactatgacttgtacaactctttcatagctgatctatcgaagatcgatagttcctatgcaagtaaggactctttaacacttacttacggttccgacaattacaaaaacgttatcaaatccttgctacaatttggtgattcctttttaaaggttttgttggatcatattgatgataatggtcaattaactgaagaaattaacagatacactggttttcaagctggcgccgtatcattgacatggtcctccggttctttgttgtctgctaatagggcaagaaacaaattaatcgagctattataa

SEQ ID NO: 4 - protein, 489 aa, unidentified
  source 1..489: note = Saccharomycopsis fibuligera
NTGHFQAYSGYTVARSNFTQWIHEQPAVSWYYLLQNIDYPEGQFKSAKPGVVVASPSTSEPDYFYQWTRDTAITFLSLIAEVEDHSFSNTTLAKVVEYYISNTYTLQRVSNPSGNFDSPNHDGLGEPKFNVDDTAYTASWGRPQNDGPALRAYAISRYLNAVAKHNNGKLLLAGQNGIPYSSASDIYWKIIKPDLQHVSTHWSTSGFDLWEENQGTHFFTALVQLKALSYGIPLSKTYNDPGFTSWLEKQKDALNSYINSSGFVNSGKKHIVESPQLSSRGGLDSATYIAALITHDIGDDDTYTPFNVDNSYVLNSLYYLLVDNKNRYKINGNYKAGAAVGRYPEDVYNGVGTSEGNPWQLATAYAGQTFYTLAYNSLKNKKNLVIEKLNYDLYNSFIADLSKIDSSYASKDSLTLTYGSDNYKNVIKSLLQFGDSFLKVLLDHIDDNGQLTEEINRYTGFQAGAVSLTWSSGSLLSANRARNKLIELL

SEQ ID NO: 5 - DNA, 1851 bp, synthetic construct
  source 1..1851: mol_type = other DNA
gccccacaattgtcccccagggctacttctctagattcctggttatccagcgaaactactttttctttgaacggtattctcgccaacatcggttcttctggtgcttactctaagtctgctgcctctggtgccgtcatcgcttccccttctactagcaaccccgattactattatacctggaccagagacgcagcgttaactttgaaagccttagttgatattttccgtaatggcaatttgggtctacaaaccgttatcgaacaatatgttaatgcacaggctaaattgcaaactgtctctaatccttccggaggtttgtccgacggtgcaggtttgggagaacctaagttcaatgttgacttgtctgctttcactggtgcttggggtagaccacaaagagatggcccggctctacgggctatagcactaatcgatttcggcaattggctgatagataacggatataaatcttacgcggtgaacaacgtttggccaatcgtaaggaacgatttggcctatgttgcccagtactggtcacagtccggcttcgacctatgggaagaagtgaattctatgtctttctttacagttgctaaccaacatcgttcattagtcgaaggatcagctttcgcatctcgtgtcggtgccagctgttctggttgtgactctcaagctcctcagattttgtgttacatgcaatctttttggactgggagttatattaatgccaatacgggtggtggtagatccggtaaagattctaacactattttagcctcgatacatacttttgatcctgctgcttcttgtgatgacgttaccttccaaccatgctcaagtagagctttggctaaccacaaggtctataccgattctttcagatccgtttacgcgttaaactccggtatagcccaaggtaaggccgtttctgtaggtcgttacccagaagatagttactacggtggcaacccatggtttttatcaaacttagcagctgctgagcaactttatgatgctatctaccaatggaacaagattggttccatcactatcacctcgacctcgcttgcatttttcaaggatgtttatccgtctgccgctaccggtacctatgcttctgggtccacaacctttaatgctattatttctgcagtaaagacatatgctgacggctatgtcagtattgttcaatcccactcctatgcgaatggttcgttgtcagaacaattcgacagaaccactggtttgtccatcagtgctcgcgatttaacatggtcttatgcggcgctgttgactgcaaatgacagaagaaatggcgttgtccctccatcgtggggcgcaagttccgctaattcgatacctggttcatgcagcatgggttctgccacaggttcctacgctactccatctgttggttcatggccagcaacacttacttcaggtacagctgcaccttccagtacatcaactactaccaaggctccaactaccaccacggccaccacaacaacttccgccggttcctgtactacaccaaccgcagtggctgttactttcgatgaaattgctacgacgacatttggtgaaaacgtctacttggtaggaagcattagccaattaggtaactggaatacagccaacggtatcccactgtctgcttcaaagtacacctcttcaaatccattatggtacgccactgtgaacttgcccgctggcactacttttcaatacaaatattttagaaaggaatctgatggttccatcaaatgggagtcagacccaaacagatcttacactgttccagccaaatgtggtactactacagccacagaaaatgatacttggagataa

SEQ ID NO: 6 - protein, 616 aa, unidentified
  source 1..616: note = Penicillium oxalicum
APQLSPRATSLDSWLSSETTFSLNGILANIGSSGAYSKSAASGAVIASPSTSNPDYYYTWTRDAALTLKALVDIFRNGNLGLQTVIEQYVNAQAKLQTVSNPSGGLSDGAGLGEPKFNVDLSAFTGAWGRPQRDGPALRAIALIDFGNWLIDNGYKSYAVNNVWPIVRNDLAYVAQYWSQSGFDLWEEVNSMSFFTVANQHRSLVEGSAFASRVGASCSGCDSQAPQILCYMQSFWTGSYINANTGGGRSGKDSNTILASIHTFDPAASCDDVTFQPCSSRALANHKVYTDSFRSVYALNSGIAQGKAVSVGRYPEDSYYGGNPWFLSNLAAAEQLYDAIYQWNKIGSITITSTSLAFFKDVYPSAATGTYASGSTTFNAIISAVKTYADGYVSIVQSHSYANGSLSEQFDRTTGLSISARDLTWSYAALLTANDRRNGVVPPSWGASSANSIPGSCSMGSATGSYATPSVGSWPATLTSGTAAPSSTSTTTKAPTTTTATTTTSAGSCTTPTAVAVTFDEIATTTFGENVYLVGSISQLGNWNTANGIPLSASKYTSSNPLWYATVNLPAGTTFQYKYFRKESDGSIKWESDPNRSYTVPAKCGTTTATENDTWR

SEQ ID NO: 7 - DNA, 11648 bp, synthetic construct
  source 1..11648: mol_type = other DNA
ccagtttttccatgctgggtttcttttcgttaatagtggtgggtaaaagaaaacgtacgaataaaatgctgaatgtagaatatcctgtaggctcattaatacacagtagaacgcagacccattcgaggggctcattggaaacacgtagtcgacattagttctagataatccgcttgatgggccacatatggtaatggcttctcgaagcagatgttacgagccgccagaacgaggcggtggcatctgcctcgcgctgttttctagcggcagagaaaacccgtggatagtttaaaccttcgagcgtcccaaaaccttctcaagcaaggttttcagtataatgttacatgcgtacacgcgtttgtacagaaaaaaaagaaaaatttgaaatataaataacgttcttaatactaacataactataaaaaaataaatagggacctagacttcaggttgtctaactccttccttttcggttagagcggatatttcgaaatctttcgattagcacgcacacacatcacatagactgcgtcataaaaatacactacggaaaaaccataaagagcaaagcgatacctacttggaaggaaaaggagcacgcttgtaagggggatgggggctaagaagtcattcactttcttttcccttcgcggtccggacccgggacccctcctctccccgcacaatttcttcctttcatatcttccttttattcctatcccgttgaagcaaccgcactatgactaaatggtgctggacatctccatggctgtgacttgtgtgtatctcacagtggtaacggcaccgtggctcggaaacggttccttcgtgacaattctagaacaggggctacagtctcgataatagaataataagcgcatttttgttagcgccgccgcggcgcccgtttcccaatagggaggcgcagtttatcggcggagctttacttcttcctatttgggtaagcccctttctgttttcggccagtggttgctgcaggctgcgccggagaacatagtgataagggatgtaactttcgatgagagaattagcaagcggaaaaaaaactatggctagctgggagttgtttttcaatcatataaaagggagaaattgttgctcactatgtgacagtttctgggacgtcttaacttttattgcagaggactatcaaatcatacagatattgtcaaaaaaaaaaaaaaagactaataataaaaaatgatcagattgactgtcttcttaaccgctgttttcgcagctgtcgcatcttgtgttcccgttgagcttgacaagagaaatacaggtcatttccaagcctactctggttacacagttgctcgttccaacttcacccaatggattcacgaacaacctgccgtgtcatggtattatttgcttcagaatattgactacccagaaggccagttcaaatcggccaagcctggtgttgttgtggccagcccatctacttcagagccagattacttttaccaatggactagagatactgcaattactttcttgagtttgattgctgaagttgaagaccattctttttcaaacactactttggctaaggtcgttgaatactacatttcaaatacatacaccttacaaagagtatcgaacccatcaggtaactttgacagcccaaaccatgatggtttaggtgaaccaaagtttaatgtggatgataccgcatatactgcttcttggggtcgtcctcaaaatgacggtccagctttgagagcttatgctatttctaggtatctgaatgccgtcgccaaacacaacaacggtaagttgctgctcgcgggccaaaacggtataccgtattcttctgcctctgatatctactggaaaattattaaacctgatttacaacatgtttccacccattggtctacctccggatttgatttgtgggaagagaaccaaggtactcacttcttcacggcactagtgcagttgaaagctctatcttatggtattcctttgtccaagacttataatgatccagggtttacctcgtggttggaaaagcaaaaggatgctttaaattcctacataaattcttccggtttcgttaattcaggcaaaaagcacattgtcgaatctccacaacttagttctagaggtggtttggactcagctacctatatcgccgctctaatcacccacgatattggtgacgatgacacctacactccattcaatgtcgacaacagctatgtcttaaacagtttatattacttattggttgataacaagaatcgttataaaatcaacggaaactacaaggctggtgctgctgttggtagatatcctgaagatgtttacaatggtgtcggaacttctgaaggtaatccatggcaattggccactgcctacgctggtcaaactttttatacattagcttacaactccttgaagaacaagaaaaatttagtaattgaaaaattgaactatgacttgtacaactctttcatagctgatctatcgaagatcgatagttcctatgcaagtaaggactctttaacacttacttacggttccgacaattacaaaaacgttatcaaatccttgctacaatttggtgattcctttttaaaggttttgttggatcatattgatgataatggtcaattaactgaagaaattaacagatacactggttttcaagctggcgccgtatcattgacatggtcctccggttctttgttgtctgctaatagggcaagaaacaaattaatcgagctattataaattgaattgaattgaaatcgatagatcaatttttttcttttctctttccccatcctttacgctaaaataatagtttattttattttttgaatattttttatttatatacgtatatatagactattatttatcttttaatgattattaagatttttattaaaaaaaaattcgctcctcttttaatgcctttatccagtttttttttcccattcgatatttctatgttcgggttcagcgtattttaagtttaataactcgaaaattctgcgttcgttaaagctttcgagaaggatattatttcgaaataaaccgtgttgtgtaagcttgaagcctttttgcgctgccaatattcttatccatctattgtactctttagatccagtatagtgtattcttcctgctccaagttcatcccacttgcaacaaaactttcgattagcacgcacacacatcacatagactgcgtcataaaaatacactacggaaaaaccataaagagcaaagcgatacctacttggaaggaaaaggagcacgcttgtaagggggatgggggctaagaagtcattcactttcttttcccttcgcggtccggacccgggacccctcctctccccgcacaatttcttcctttcatatcttccttttattcctatcccgttgaagcaaccgcactatgactaaatggtgctggacatctccatggctgtgacttgtgtgtatctcacagtggtaacggcaccgtggctcggaaacggttccttcgtgacaattctagaacaggggctacagtctcgataatagaataataagcgcatttttgttagcgccgccgcggcgcccgtttcccaatagggaggcgcagtttatcggcggagctttacttcttcctatttgggtaagcccctttctgttttcggccagtggttgctgcaggctgcgccggagaacatagtgataagggatgtaactttcgatgagagaattagcaagcggaaaaaaaactatggctagctgggagttgtttttcaatcatataaaagggagaaattgttgctcactatgtgacagtttctgggacgtcttaacttttattgcagaggactatcaaatcatacagatattgtcaaaaaaaaaaaaaaagactaataataaaaaatgatcagattgactgtcttcttaaccgctgttttcgcagctgtcgcatcttgtgttcccgttgagcttgacaagagaaatacaggtcatttccaagcctactctggttacacagttgctcgttccaacttcacccaatggattcacgaacaacctgccgtgtcatggtattatttgcttcagaatattgactacccagaaggccagttcaaatcggccaagcctggtgttgttgtggccagcccatctacttcagagccagattacttttaccaatggactagagatactgcaattactttcttgagtttgattgctgaagttgaagaccattctttttcaaacactactttggctaaggtcgttgaatactacatttcaaatacatacaccttacaaagagtatcgaacccatcaggtaactttgacagcccaaaccatgatggtttaggtgaaccaaagtttaatgtggatgataccgcatatactgcttcttggggtcgtcctcaaaatgacggtccagctttgagagcttatgctatttctaggtatctgaatgccgtcgccaaacacaacaacggtaagttgctgctcgcgggccaaaacggtataccgtattcttctgcctctgatatctactggaaaattattaaacctgatttacaacatgtttccacccattggtctacctccggatttgatttgtgggaagagaaccaaggtactcacttcttcacggcactagtgcagttgaaagctctatcttatggtattcctttgtccaagacttataatgatccagggtttacctcgtggttggaaaagcaaaaggatgctttaaattcctacataaattcttccggtttcgttaattcaggcaaaaagcacattgtcgaatctccacaacttagttctagaggtggtttggactcagctacctatatcgccgctctaatcacccacgatattggtgacgatgacacctacactccattcaatgtcgacaacagctatgtcttaaacagtttatattacttattggttgataacaagaatcgttataaaatcaacggaaactacaaggctggtgctgctgttggtagatatcctgaagatgtttacaatggtgtcggaacttctgaaggtaatccatggcaattggccactgcctacgctggtcaaactttttatacattagcttacaactccttgaagaacaagaaaaatttagtaattgaaaaattgaactatgacttgtacaactctttcatagctgatctatcgaagatcgatagttcctatgcaagtaaggactctttaacacttacttacggttccgacaattacaaaaacgttatcaaatccttgctacaatttggtgattcctttttaaaggttttgttggatcatattgatgataatggtcaattaactgaagaaattaacagatacactggttttcaagctggcgccgtatcattgacatggtcctccggttctttgttgtctgctaatagggcaagaaacaaattaatcgagctattataaattgaattgaattgaaatcgatagatcaatttttttcttttctctttccccatcctttacgctaaaataatagtttattttattttttgaatattttttatttatatacgtatatatagactattatttatcttttaatgattattaagatttttattaaaaaaaaattcgctcctcttttaatgcctttatccagtttttttttcccattcgatatttctatgttcgggttcagcgtattttaagtttaataactcgaaaattctgcgttcgttaaagctttcgagaaggatattatttcgaaataaaccgtgttgtgtaagcttgaagcctttttgcgctgccaatattcttatccatctattgtactctttagatccagtatagtgtattcttcctgctccaagttcatcccacttgcaacaaaactttcgattagcacgcacacacatcacatagactgcgtcataaaaatacactacggaaaaaccataaagagcaaagcgatacctacttggaaggaaaaggagcacgcttgtaagggggatgggggctaagaagtcattcactttcttttcccttcgcggtccggacccgggacccctcctctccccgcacaatttcttcctttcatatcttccttttattcctatcccgttgaagcaaccgcactatgactaaatggtgctggacatctccatggctgtgacttgtgtgtatctcacagtggtaacggcaccgtggctcggaaacggttccttcgtgacaattctagaacaggggctacagtctcgataatagaataataagcgcatttttgttagcgccgccgcggcgcccgtttcccaatagggaggcgcagtttatcggcggagctttacttcttcctatttgggtaagcccctttctgttttcggccagtggttgctgcaggctgcgccggagaacatagtgataagggatgtaactttcgatgagagaattagcaagcggaaaaaaaactatggctagctgggagttgtttttcaatcatataaaagggagaaattgttgctcactatgtgacagtttctgggacgtcttaacttttattgcagaggactatcaaatcatacagatattgtcaaaaaaaaaaaaaaagactaataataaaaaatgatcagattgactgtcttcttaaccgctgttttcgcagctgtcgcatcttgtgttcccgttgagcttgacaagagaaatacaggtcatttccaagcctactctggttacacagttgctcgttccaacttcacccaatggattcacgaacaacctgccgtgtcatggtattatttgcttcagaatattgactacccagaaggccagttcaaatcggccaagcctggtgttgttgtggccagcccatctacttcagagccagattacttttaccaatggactagagatactgcaattactttcttgagtttgattgctgaagttgaagaccattctttttcaaacactactttggctaaggtcgttgaatactacatttcaaatacatacaccttacaaagagtatcgaacccatcaggtaactttgacagcccaaaccatgatggtttaggtgaaccaaagtttaatgtggatgataccgcatatactgcttcttggggtcgtcctcaaaatgacggtccagctttgagagcttatgctatttctaggtatctgaatgccgtcgccaaacacaacaacggtaagttgctgctcgcgggccaaaacggtataccgtattcttctgcctctgatatctactggaaaattattaaacctgatttacaacatgtttccacccattggtctacctccggatttgatttgtgggaagagaaccaaggtactcacttcttcacggcactagtgcagttgaaagctctatcttatggtattcctttgtccaagacttataatgatccagggtttacctcgtggttggaaaagcaaaaggatgctttaaattcctacataaattcttccggtttcgttaattcaggcaaaaagcacattgtcgaatctccacaacttagttctagaggtggtttggactcagctacctatatcgccgctctaatcacccacgatattggtgacgatgacacctacactccattcaatgtcgacaacagctatgtcttaaacagtttatattacttattggttgataacaagaatcgttataaaatcaacggaaactacaaggctggtgctgctgttggtagatatcctgaagatgtttacaatggtgtcggaacttctgaaggtaatccatggcaattggccactgcctacgctggtcaaactttttatacattagcttacaactccttgaagaacaagaaaaatttagtaattgaaaaattgaactatgacttgtacaactctttcatagctgatctatcgaagatcgatagttcctatgcaagtaaggactctttaacacttacttacggttccgacaattacaaaaacgttatcaaatccttgctacaatttggtgattcctttttaaaggttttgttggatcatattgatgataatggtcaattaactgaagaaattaacagatacactggttttcaagctggcgccgtatcattgacatggtcctccggttctttgttgtctgctaatagggcaagaaacaaattaatcgagctattataaattgaattgaattgaaatcgatagatcaatttttttcttttctctttccccatcctttacgctaaaataatagtttattttattttttgaatattttttatttatatacgtatatatagactattatttatcttttaatgattattaagatttttattaaaaaaaaattcgctcctcttttaatgcctttatccagtttttttttcccattcgatatttctatgttcgggttcagcgtattttaagtttaataactcgaaaattctgcgttcgttaaagctttcgagaaggatattatttcgaaataaaccgtgttgtgtaagcttgaagcctttttgcgctgccaatattcttatccatctattgtactctttagatccagtatagtgtattcttcctgctccaagttcatcccacttgcaacaaaactttcgattagcacgcacacacatcacatagactgcgtcataaaaatacactacggaaaaaccataaagagcaaagcgatacctacttggaaggaaaaggagcacgcttgtaagggggatgggggctaagaagtcattcactttcttttcccttcgcggtccggacccgggacccctcctctccccgcacaatttcttcctttcatatcttccttttattcctatcccgttgaagcaaccgcactatgactaaatggtgctggacatctccatggctgtgacttgtgtgtatctcacagtggtaacggcaccgtggctcggaaacggttccttcgtgacaattctagaacaggggctacagtctcgataatagaataataagcgcatttttgttagcgccgccgcggcgcccgtttcccaatagggaggcgcagtttatcggcggagctttacttcttcctatttgggtaagcccctttctgttttcggccagtggttgctgcaggctgcgccggagaacatagtgataagggatgtaactttcgatgagagaattagcaagcggaaaaaaaactatggctagctgggagttgtttttcaatcatataaaagggagaaattgttgctcactatgtgacagtttctgggacgtcttaacttttattgcagaggactatcaaatcatacagatattgtcaaaaaaaaaaaaaaagactaataataaaaaatgatcagattgactgtcttcttaaccgctgttttcgcagctgtcgcatcttgtgttcccgttgagcttgacaagagagccccacaattgtcccccagggctacttctctagattcctggttatccagcgaaactactttttctttgaacggtattctcgccaacatcggttcttctggtgcttactctaagtctgctgcctctggtgccgtcatcgcttccccttctactagcaaccccgattactattatacctggaccagagacgcagcgttaactttgaaagccttagttgatattttccgtaatggcaatttgggtctacaaaccgttatcgaacaatatgttaatgcacaggctaaattgcaaactgtctctaatccttccggaggtttgtccgacggtgcaggtttgggagaacctaagttcaatgttgacttgtctgctttcactggtgcttggggtagaccacaaagagatggcccggctctacgggctatagcactaatcgatttcggcaattggctgatagataacggatataaatcttacgcggtgaacaacgtttggccaatcgtaaggaacgatttggcctatgttgcccagtactggtcacagtccggcttcgacctatgggaagaagtgaattctatgtctttctttacagttgctaaccaacatcgttcattagtcgaaggatcagctttcgcatctcgtgtcggtgccagctgttctggttgtgactctcaagctcctcagattttgtgttacatgcaatctttttggactgggagttatattaatgccaatacgggtggtggtagatccggtaaagattctaacactattttagcctcgatacatacttttgatcctgctgcttcttgtgatgacgttaccttccaaccatgctcaagtagagctttggctaaccacaaggtctataccgattctttcagatccgtttacgcgttaaactccggtatagcccaaggtaaggccgtttctgtaggtcgttacccagaagatagttactacggtggcaacccatggtttttatcaaacttagcagctgctgagcaactttatgatgctatctaccaatggaaaaagattggttccatcactatcacctcgacctcgcttgcatttttcaaggatgtttatccgtctgccgctaccggtacctatgcttctgggtccacaacctttaatgctattatttctgcagtaaagacatatgctgacggctatgtcagtattgttcaatcccactcctatgcgaatggttcgttgtcagaacaattcgacagaaccactggtttgtccatcagtgctcgcgatttaacatggtcttatgcggcgctgttgactgcaaatgacagaagaaatggcgttgtccctccatcgtggggcgcaagttccgctaattcgatacctggttcatgcagcatgggttctgccacaggttcctacgctactccatctgttggttcatggccagcaacacttacttcaggtacagctgcaccttccagtacatcaactactaccaaggctccaactaccaccacggccaccacaacaacttccgccggttcctgtactacaccaaccgcagtggctgttactttcgatgaaattgctacgacgacatttggtgaaaacgtctacttggtaggaagcattagccaattaggtaactggaatacagccaacggtatcccactgtctgcttcaaagtacacctcttcaaatccattatggtacgccactgtgaacttgcccgctggcactacttttcaatacaaatattttagaaaggaatctgatggttccatcaaatgggagtcagacccaaacagatcttacactgttccagccaaatgtggtactactacagccacagaaaatgatacttggagataaatttaaatgtagatacgttgttgacacttctaaataagcgaatttcttatgatttatgatttttattattaaataagttataaaaaaaataagtgtatacaaattttaaagtgactcttaggttttaaaacgaaaattcttattcttgagtaactctttcctgtaggtcaggttgctttctcaggtatagcatgaggtcgctcgtttaaacgaatttcgttgtcacgttgttttggtaagttccttcgctttctcgtaaaaataagtaaaaatccggggaaactattatttgcggttcgaaataaaagcattataatttccttccttggcacatttcttggccacggatgacctaaaacattgccaaataaaaaggggtaagagaactt

SEQ ID NO: 8 - DNA, 7552 bp, synthetic construct
  source 1..7552: mol_type = other DNA
ccattttgagcgagagaacccatttttctatacaaatttcactagagcacggccgttacatttagtaatagccaataagggttttttatcgattagtgttccctgcgctccttaacatcatacaaccgagtccttgacatggaaatagtaggcaagtaaaccaaagtcctttcttcaaaagtagaaaacttgagcacttatttcctgcgcatgtcatatgttaattttccttaactgcgctgaatacgtcctgtcaattcaaatatatcacgttttgagcagccctaaagaagaaaacctcaacagcagtattactattacaatcaaacaactttagtgccgcgtgataccgggggttgaagtgggtgcattgagccgtattcttcttccccgtaagaaagttatgtatcctttttactgcgttgtaatagcttctgaaaacctaaaaaatgaacgctatgtagctcatatccgtttcgcataagtaagaataactacttgtttaaaccttcgagcgtcccaaaaccttctcaagcaaggttttcagtataatgttacatgcgtacacgcgtttgtacagaaaaaaaagaaaaatttgaaatataaataacgttcttaatactaacataactataaaaaaataaatagggacctagacttcaggttgtctaactccttccttttcggttagagcggatatttcgaaatctttcgattagcacgcacacacatcacatagactgcgtcataaaaatacactacggaaaaaccataaagagcaaagcgatacctacttggaaggaaaaggagcacgcttgtaagggggatgggggctaagaagtcattcactttcttttcccttcgcggtccggacccgggacccctcctctccccgcacaatttcttcctttcatatcttccttttattcctatcccgttgaagcaaccgcactatgactaaatggtgctggacatctccatggctgtgacttgtgtgtatctcacagtggtaacggcaccgtggctcggaaacggttccttcgtgacaattctagaacaggggctacagtctcgataatagaataataagcgcatttttgttagcgccgccgcggcgcccgtttcccaatagggaggcgcagtttatcggcggagctttacttcttcctatttgggtaagcccctttctgttttcggccagtggttgctgcaggctgcgccggagaacatagtgataagggatgtaactttcgatgagagaattagcaagcggaaaaaaaactatggctagctgggagttgtttttcaatcatataaaagggagaaattgttgctcactatgtgacagtttctgggacgtcttaacttttattgcagaggactatcaaatcatacagatattgtcaaaaaaaaaaaaaaagactaataataaaaaatgatcagattgactgtcttcttaaccgctgttttcgcagctgtcgcatcttgtgttcccgttgagcttgacaagagagattcatacaccacctcaacagacgattcgtctaatgacactgccgacagtgtctctgatggtgtgattttacacgcttggtgttggtctttcaacacaatcaagaacaatttgaagcaaattcacgatgcaggttacactgccgttcaaacctcccctgtcaatgaagtcaaagttggtaattctgctagtaagtctttgaacaactggtactggttataccaaccaacaaagtactcgattggtaactattacttaggtaccgaagctgaattcaagtccatgtgtgcagctgccaaggagtacaacatcagaattattgttgatgctaccttgaatgacaccacaagtgactactcagctatttcggatgaaatcaaatccattagtaattggactcatggcaatacacagatatccaactggtcagacagggaggatgtcacccaaaactctctccttggtttgtatgattggaacactcaaaattcccaagtccaaacatacctaaagaactacttggaacgtctaatatcagatggggcaagcggttttcgttacgatgcagccaaacatatcgaattgccatcacaatacgacggttcatatggttccaatttttggccaaatatcactgacaatggtagtgaattccaatatggcgaagttttgcaagattctatttccaaagaatccgattacgctaattacatgtcagtaacagcctctaattatggtaatactattagaaatgccctgaaaaacagagatttcactgctagcacattacaaaatttcaatatttctgtccccgctagcaagttggttacttgggttgaatctcatgacaactatgcaaacgatgaccaagtttctacctggatgaatagttccgatattaaactaggttgggccgtagtggcctcaagatctggaagtgttccattatttttcgacagaccagttgacggtggtaatggtacccgttttcctggatctagcgaaattggtgacgccggttcttcgctttattatgacaaggctgttgtggcggttaacaagttccacaacgccatggctggtcaatctgaatacatttcaaacccaaacggtaacaccaaaatttttgaaaacgaaagaggttctaagggtgtcgttttcgctaatgcttcggatggcagctattctctatctgttaagacatctcttgctgacggtacctacgaaaataaggccggaagtgacgagttcactgttaaaaacggttatttgacaggtactatccaaggtagagaagtagtcgtattatatggcgatccaacttcaagctcgtcctcgtctaccactactgaaactaagaaggtgtattttgaaaaaccatcctcctggggttccacagtctatgcctatgtctacaacaaaaacactaataaggctataaccagcgcatggccaggtaaagagatgactgctttaggtaatgatgagtataaattagacctggatacagatgaagatgattccgacttggcagtaattttcaccgatgggaccaaccaaactcctgcagccaacaaggctgggttcaccttcacagcagacgcgacgtacgatcagaacggtgttgttaagacctctgactcatcttcgtcgtcctccactaccaccgaaacaaaaaaagtgtattttgaaaagccttcatcttgggggtccactgtctacgcctacgtttataataaaaacacgaacaaagctatcaccagtgcttggcccggtaaggaaatgaccgctcttggaaatgacgaatataaattggatttggatactgatgaagatgatagtgatctagctgttatctttactgatggtacaaaccaaacgccggcagctaacaaggcaggtttcacttttaccgctgatgccacttatgatcaaaacggtgtggttaagacatctgacagttcttcatcatcttccagtacaactacggaaactaagaaagtttacttcgaaaagccatcttcgtggggctctacggtttacgcttatgtttataacaagaatacaaataaagcaattacttccgcttggcctggtaaggaaatgactgcgttaggcaacgacgaatacaagttagatttagataccgatgaagatgatagtgatttggctgtgatcttcactgatggaaccaaccagactccagctgctaacaaagcaggctttacctttactgctgatgccacttatgaccagaatggtgttgtcaagacctccgatagctcctcttcctcgtcaactactacagaaacgaagaaggtttactttgagaagccaagtagttggggttctacagtttatgcttacgtatacaataaaaatactaataaagcgatcactagcgcctggccaggtaaagaaatgacagctttgggcaatgacgaatacaaattggaccttgacactgacgaggacgactccgatttggctgttatatttaccgatggtactaatcaaacgcctgctgcaaataaagctggtttcacatttaccgccgatgctacttacgatcagaacggtgtcgtcaaaacatctgattcttcgtccacctcttctacatcataaattgaattgaattgaaatcgatagatcaatttttttcttttctctttccccatcctttacgctaaaataatagtttattttattttttgaatattttttatttatatacgtatatatagactattatttatcttttaatgattattaagatttttattaaaaaaaaattcgctcctcttttaatgcctttatccagtttttttttcccattcgatatttctatgttcgggttcagcgtattttaagtttaataactcgaaaattctgcgttcgttaaagctttcgagaaggatattatttcgaaataaaccgtgttgtgtaagcttgaagcctttttgcgctgccaatattcttatccatctattgtactctttagatccagtatagtgtattcttcctgctccaagttcatcccacttgcaacaaaactttcgattagcacgcacacacatcacatagactgcgtcataaaaatacactacggaaaaaccataaagagcaaagcgatacctacttggaaggaaaaggagcacgcttgtaagggggatgggggctaagaagtcattcactttcttttcccttcgcggtccggacccgggacccctcctctccccgcacaatttcttcctttcatatcttccttttattcctatcccgttgaagcaaccgcactatgactaaatggtgctggacatctccatggctgtgacttgtgtgtatctcacagtggtaacggcaccgtggctcggaaacggttccttcgtgacaattctagaacaggggctacagtctcgataatagaataataagcgcatttttgttagcgccgccgcggcgcccgtttcccaatagggaggcgcagtttatcggcggagctttacttcttcctatttgggtaagcccctttctgttttcggccagtggttgctgcaggctgcgccggagaacatagtgataagggatgtaactttcgatgagagaattagcaagcggaaaaaaaactatggctagctgggagttgtttttcaatcatataaaagggagaaattgttgctcactatgtgacagtttctgggacgtcttaacttttattgcagaggactatcaaatcatacagatattgtcaaaaaaaaaaaaaaagactaataataaaaaatgatcagattgactgtcttcttaaccgctgttttcgcagctgtcgcatcttgtgttcccgttgagcttgacaagagaaatacaggtcatttccaagcctactctggttacacagttgctcgttccaacttcacccaatggattcacgaacaacctgccgtgtcatggtattatttgcttcagaatattgactacccagaaggccagttcaaatcggccaagcctggtgttgttgtggccagcccatctacttcagagccagattacttttaccaatggactagagatactgcaattactttcttgagtttgattgctgaagttgaagaccattctttttcaaacactactttggctaaggtcgttgaatactacatttcaaatacatacaccttacaaagagtatcgaacccatcaggtaactttgacagcccaaaccatgatggtttaggtgaaccaaagtttaatgtggatgataccgcatatactgcttcttggggtcgtcctcaaaatgacggtccagctttgagagcttatgctatttctaggtatctgaatgccgtcgccaaacacaacaacggtaagttgctgctcgcgggccaaaacggtataccgtattcttctgcctctgatatctactggaaaattattaaacctgatttacaacatgtttccacccattggtctacctccggatttgatttgtgggaagagaaccaaggtactcacttcttcacggcactagtgcagttgaaagctctatcttatggtattcctttgtccaagacttataatgatccagggtttacctcgtggttggaaaagcaaaaggatgctttaaattcctacataaattcttccggtttcgttaattcaggcaaaaagcacattgtcgaatctccacaacttagttctagaggtggtttggactcagctacctatatcgccgctctaatcacccacgatattggtgacgatgacacctacactccattcaatgtcgacaacagctatgtcttaaacagtttatattacttattggttgataacaagaatcgttataaaatcaacggaaactacaaggctggtgctgctgttggtagatatcctgaagatgtttacaatggtgtcggaacttctgaaggtaatccatggcaattggccactgcctacgctggtcaaactttttatacattagcttacaactccttgaagaacaagaaaaatttagtaattgaaaaattgaactatgacttgtacaactctttcatagctgatctatcgaagatcgatagttcctatgcaagtaaggactctttaacacttacttacggttccgacaattacaaaaacgttatcaaatccttgctacaatttggtgattcctttttaaaggttttgttggatcatattgatgataatggtcaattaactgaagaaattaacagatacactggttttcaagctggcgccgtatcattgacatggtcctccggttctttgttgtctgctaatagggcaagaaacaaattaatcgagctattataaatttaaatgtagatacgttgttgacacttctaaataagcgaatttcttatgatttatgatttttattattaaataagttataaaaaaaataagtgtatacaaattttaaagtgactcttaggttttaaaacgaaaattcttattcttgagtaactctttcctgtaggtcaggttgctttctcaggtatagcatgaggtcgctcgtttaaacaaaaccgctgcagcaacccttgttacatacagtcggatccatctgacttactttccttgcgtctccctgcgcgatcttgttggccattttccagatcctctagaatttttcaagggtcgagccgtaggaggattctctcagaaggcaaaaacgcatcgaaagcgtgctttgtaagaatatttggtatggctaaagtaagcaaagccatatcccgatcccgatcccgactcttattccgatcccttctgccacatcctgcatgtttattcgaataccgaattagctcatcttcgttattttcatcatccctttctgctatagcaaggacaagtttttttctagcatctcatcgaaaactttcctctccctaattggccaaagttttcatattcatcatcagttagaaagtataatatcaatcccttacctcattacaagttgtatcacactaaaaaaatcatatataagtctgtgagagtcttcaattatttagcgtaaca

(19) United States
(12) Patent Application Publication
(10) Pub. No.: US 2024/0336929 A1
(43) Pub. Date: Oct. 10, 2024

(54) STRAINS OF SACCHAROMYCES CEREVISIAE THAT EXHIBIT AN INCREASED ABILITY TO FERMENT OLIGOSACCHARIDES INTO ETHANOL WITHOUT SUPPLEMENTAL GLUCOAMYLASE AND METHODS OF MAKING AND USING THE SAME

(71) Applicant: Xylogenics, Inc., Brownsburg, IN (US)

(72) Inventors: Joshua W. Heyen, Brownsburg, IN (US); Nadaraj Palaniappan, Carmel, IN (US); Kathryn A. Houin, Lebanon, IN (US); Ross Cocklin, Keene, NH (US); Mark G. Goebl, Indianapolis, IN (US)

(21) Appl. No.: 18/579,192

(22) PCT Filed: Jul. 12, 2022
(86) PCT No.: PCT/US22/73659

§ 371 Date: Jan. 12, 2024

Related U.S. Application Data
(60) Provisional application No. 63/220,930, filed on Jul. 12, 2021.

(51) Int. Cl. C12N 15/81; C12N 9/28; C12N 9/34; C12N 15/52; C12P 7/06; C12R 1/25; C12R 1/80; C12R 1/865

(52) U.S. Cl. CPC C12N 15/81; C12N 9/2417; C12N 9/2428; C12N 15/52; C12P 7/06; C12N 2510/02; C12R 2001/25; C12R 2001/80; C12R 2001/865

(57) ABSTRACT

Disclosed herein is a yeast strain capable of fermenting corn mash into ethanol with no exogenous glucoamy lase resulting in greater speed and efficiency than the leading industrial strain. Said yeast strain causes enhanced co-fermentation of DP3 sugars, maltose and glucose in strains otherwise identical to commercial fuel ethanol yeast strains.

### PRIORITY CLAIM

This application claims the benefit of U.S. provisional patent application No. 63/220,930, filed on Jul. 12, 2021, the disclosure of which in incorporated herein by reference in its entirety.

### REFERENCE TO SEQUENCE LISTING

The instant application contains a Sequence Listing XML which has been submitted electronically and is hereby incorporated by reference in its entirety. Said Sequence Listing XML copy, created on Jul. 12, 2022, is named XYLO-0003-01-WO.xml and is 35,007 bytes in size.

### FIELD OF THE INVENTION

Aspects of the invention relate to making and using strains of Saccharomyces cerevisiae that are capable of rapidly and efficiently fermenting corn mash into ethanol using an endogenously expressed maltogenic alpha-amylase, multiple types and copies of glucoamylases, all in a strain constructed to co-consume maltose and glucose; thereby, either eliminating or reducing the need to convert disaccharides and trisaccharides into glucose through the addition of glucoamylase enzymes to yeast feed stocks.

### BACKGROUND

Various species of Saccharomyces are among the most important industrially grown microorganisms. Long used to leaven bread, produce beer and wine, and as a source of food flavorings and micronutrients, these organisms now play a central role in the production of fuel, facilitating the conversion of sugars to ethanol. A metabolically complex organism, yeast can grow both aerobically and anaerobically as well, if certain nutritional conditions are met. When grown commercially, as in the production of yeast used to support the commercial baking industry, yeasts such as Saccharomyces cerevisiae are grown in highly aerated fermentation tanks. The growth of yeast under these conditions is manipulated to favor the production of yeast biomass. One way in which this is accomplished is to schedule the addition of sugars, such as D-glucose, and the rate of oxygen transfer to the yeast to encourage aerobic growth. Various strains of Saccharomyces can also be grown under conditions designed to maximize the production of ethanol. Oftentimes, when the object is to maximize the conversion of sugar to ethanol, the level of oxygen in the fermentation vessel is reduced relative to the levels of oxygen used in the vessel during yeast biomass production in order to favor anaerobic growth.

Most strains of Saccharomyces prefer growth on D-glucose although many strains are known to grow on other naturally occurring hexoses and even some disaccharides as well. The ability of different species of Saccharomyces to grow on different sugars and in the presence of different levels of oxygen accounts for much of its commercial utility including the central role that yeast currently plays in the conversion of plant bio-mass into ethanol for various uses including its use as a fuel.

One of the best-known pathways for the production of ethanol by yeast is the fermentation of 6-carbon sugars (hexoses) into ethanol, especially D-glucose. One widely used feedstock for the production of ethanol is the polysaccharide starch. Starch is a simple polymer consisting of chains of D-glucose. Currently, in the United States at least, starch derived from corn kernels is the preferred feed stock for bio-ethanol production by Saccharomyces cerevisiae.

A single kernel of corn is comprised of ˜65-80% starch depending on the growing season and the specific corn variety. Starch in its most basic form is a polymer of many glucose molecules linked through glycosidic bonds. This polymer can take on two basic forms. Amylose is primarily a linear glucose polymer that can contain up to 600 glucose molecules (known as DP or degree of polymerization) linked together by α-(1,4) linkages. Amylopectin however consists of large highly branched glucose polymers that can range in degree of polymerization from hundreds of thousands to millions of glucose units. Glucose units in amylopectin are linked together by both α-(1,4) and α-(1,6) linkages with the latter type providing the branching structure. Together, many amylose and amylopectin molecules intertwine into an ordered superstructure known as a starch granule (looks much like a very small onion with concentric layers). A single kernel of corn contains many starch granules consisting of 70-80% amylopectin and 20-30% amylose.

Starch granules serve to store chemical energy for the seed in a very compact and recalcitrant state. This allows for a large amount of energy to be packed into a small space while inhibiting the use of this energy reserve by microbes. In this form, starch is unavailable to the cells of the seed for energy and must therefore be broken down by enzymes into metabolizable molecules (monosaccharide and disaccharide sugars, i.e. glucose and maltose). The initial steps in producing fuel ethanol from corn are designed to achieve the same goal; breakdown of corn starch to usable cellular energy. However, the cellular energy is being used for fermentation by yeast and converted into ethanol.

The process to extract and hydrolyze corn starch in preparation for yeast fermentation starts when corn is received at the ethanol production facility. Corn is received either directly from the farmer or through other intermediaries at the ethanol plant by rail or truck. Each shipment is tested for quality by monitoring percent moisture, percent foreign particles, and the presence of toxins. Each facility has its own corn standards that must be met to accept a certain corn shipment. Corn of low moisture<=20%, low foreign particles, and minimal toxicity enables the most efficient and highest yielding fermentations. However, corn qualities such as percentage starch content, protein content, the amylose to amylopectin ratio, as well as a multitude of other factors drastically affect fermentation yield. These factors vary by region, corn hybrid, weather, farm practices, and other unpredictable variables. It is therefore common to have drastic swings in ethanol plant productivity due to variation in the corn quality from different harvests.

Once corn has been purchased and received, it is either stored on sight or fed directly to a mill. There are two different milling procedures utilized in the United States known as wet milling or dry milling. Over 70% of the 13.3 Billion gallons of fuel ethanol made in the United States in 2012 was made using what is called a dry milling or dry grind process. For this reason, the application includes-dry milling although the invention disclosed herein can be used with feed stocks prepared by virtually any milling process.

The milling process includes forming the corn into fine flour using any number of milling technologies. The most common mill utilized is a hammer mill that disrupts and grinds the corn kernel using sharpened shafts (hammers) spinning at high speed around a central axis (think enclosed fan). As the hammers spin they grind corn entering from the top of the mill until the corn is ground small enough to pass through a screen of a given size. Screen size dictates the particle size of the flour and influences many downstream processes. As flour particle size rises, the downstream enzymatic hydrolysis of the starch becomes less and less complete, ultimately decreasing the amount of sugar available to yeast and the amount of ethanol that can be produced from a given amount of corn. However, creating smaller particle sizes requires more work (energy) as the hammer mill must operate at a higher amperage to breakdown the particles. Smaller particle sizes also increase soluble solids in thin stillage, reducing centrifuge and evaporator efficiency during co-product feed production (Evaporation is an energy intensive process). For these reasons, milling practices vary across ethanol production facilities; on particles with an average screen sizes between 2.5 and 3 mm are utilized.

The ground corn flour is then mixed with water at a certain ratio in a slurry mixer. The ratio of water to corn flour determines the solids level of the final fermentation corn mash. The solids level is an important parameter in fuel ethanol production. This ratio ultimately determines the amount of sugar that is supplied to the yeast and therefore determines the maximum ethanol titer that can be achieved when the material is fermented. Today ethanol producers in the United States typically favor a 32% corn flour mixture (32% Solids) but solids levels can vary between 28 and 34%, depending on facility and season. Fermentations carried out at these solids levels are known as VHG fermentations (for Very High Gravity). The ability to carry out VHG fermentations drastically increases the efficiency of fuel ethanol production but is currently limited to the aforementioned solids levels for several reasons.

In a typical process to produce ethanol from corn the corn flour and water slurry is mixed with an α-amylase enzyme in a slurry mixer. The enzyme/corn/water mixture (mash) is then pumped to a slurry tank where it is heated to ˜90° C. to gelatinize the starch for hydrolysis by the α-amylase. The α-amylase is an endoenzyme and thus hydrolyzes glycosidic bonds within the starch granule. This action quickly reduces the viscosity of the mash as it de-polymerizes the starch polymer into shorter chain dextrins. Typically, the mash is held in the slurry tank for ˜ 20 minutes and is then sterilized, further gelatinized, and sheared in a jet cooker at 200° C. Jet cooked mash is then pumped into the liquefaction tanks, treated with a second dose of α-amylase, and held at 80-90° C. for two hours to further break down the starch into dextrins. The mash is then cooled to 30-34° C. and pumped into an 800,000 gallon fermentation tank along with yeast, nutrients, and a second enzyme, glucoamylase, to start a process known as SSF (Simultaneous Saccharification and Fermentation). Glucoamylase is an exo-acting β-amylase that liberates glucose from the non-reducing ends of starch polymers and dextrins. Thus, gluco-amylase ‘spoon feeds’ fermentable sugars to the yeast for fermentation to ethanol. The upstream processing required to produce fermentable sugars from starch for yeast fermentation is time and energy intensive.

Most commonly used glucoamylase enzyme technologies are designed to produce glucose from corn starch at a rate consistent with the rate that yeast will ferment glucose, which is preferred by normal yeast for fermentation. This preference is defined in part by the fact that when presented with a mixture of fermentable sugars, strains of Saccharomyces cerevisiae used to produce ethanol ferment glucose first and almost exclusively until virtually all the available glucose is fully consumed. Only after virtually all of glucose is completely consumed, will these strains of yeast switch to fermenting other sugars that may be available in the feed stock.

All the glucoamylase enzymes commonly used in the fuel ethanol industry are inhibited to various degrees by the presence of maltose; and maltose is almost always produced to some degree during the breakdown of starch. The accumulation of glucose in the fermenter is also undesirable as it increases the osmolarity of the environment in the fermentation vessel. Most strains of yeast used to produce ethanol are sensitive to the osmolarity of the fermentation environment; high osmolarity can reduce the efficiency of the fermentation and slow or even inhibit the ability of the yeast to produce ethanol. Accordingly, coordinating the rate of glucose production from the breakdown with the rate of glucose consumption by yeast is also necessitated by the need to reduce osmolality of the fermentation environment.

Because the accumulation of high concentrations of glucose in the fermenter broth may lead to stuck fermentations and tremendous yield reductions, traditional fermentation systems limit the rate of starch breakdown to coincide with the rate of yeast glucose fermentation. This limitation reduces the amount of starch that can be broken down and fermented in each 54-hour fermentation and thus limits maximum fermenter yield. Interestingly, maltose, which is also a fermentable sugar that can be produced from corn starch, is half as osmotically stressful to yeast and thus can accumulate to concentrations that are twice the acceptable glucose concentration in a fermenter. Therefore, the rate of starch breakdown can be greatly accelerated by producing the less stressful sugar maltose. Maltose production allows for higher solids to be loaded into a fermenter leading to higher ethanol titers, lower water usage, lower heat usage, and greater margins.

However, maltose fermentation in standard commercial yeast is glucose repressed and thus the efficiency of maltose fermentations is greatly inhibited by the accumulation of even small amounts of glucose in the fermenter using traditional commercial yeast. Thus, glucose repression has prevented the application of high gravity maltose fermentations. Some aspects of the present invention address the apparent difficulties of high gravity maltose fermentations.

### SUMMARY OF THE INVENTION

Various strains of Saccharomyces cerevisiae are the industry standard strain for commercial production of fuel ethanol from grains such as corn. One widely used strain of S. cerevisiae is the commercially available strain Ethanol Red. This strain has a robust system for utilizing glucose and includes a functional MAL2 locus which enables the strain to ferment maltose. Aspects of the present invention consists of a modified strain of Ethanol Red in which maltose and DP3 sugar fermentation has been modestly improved and glucose fermentation rates have increased, thereby improving fermentation of high maltose syrups and maltose/glucose mixtures and furthermore reducing the requirement for exogenous glucoamylase enzyme. One such example that improves maltose and glucose co-consumption through modification of the maltose uptake system is further discussed and described in U.S. patent application Ser. No. 17/261,454, filed on Jan. 19, 2021 One iteration of said example was strain ER-19-11-4. In one embodiment of the present invention, the ER-19-11-4 strain was modified to also contain a maltogenic alpha amylase along with multiple types and copies of glucoamylases. All amylase genes were codon optimized for best expression in Saccharomyces cerevisiae. The maltogenic alpha amylase is carried on the same cassette as one copy of a Saccharomycopsis fibuligera glucoamylase. We refer to this whole cassette as HMHG (SEQ ID NO: 8) Three other copies of the Saccharomycopsis fibuligera glucoamylase along with one copy of the Penicillium oxalicum glucoamylase make up what we refer to as the HGHP cassette (SEQ ID NO: 7). In this embodiment, the HGHP cassette has been integrated into the genome of ER-19-11-4 within a region encoding the Dubious Open Reading Frame YMR082C. The HMHG cassette has been integrated into the genome of ER-19-11-4 within a region encoding the Dubious Open Reading Frame YMR022C. Together, these genetic additions improve the speed and efficiency of fermentation and fully eliminate the requirement for exogenous glucoamylase, thereby significantly reducing fermentation time and material cost.

In another embodiment, the maltogenic alpha amylase is not identical to SEQ ID NO: 1 but its encoded protein products share 95% similarity with the protein products encoded in SEQ ID NO: 1 and shown as SEQ ID NO:2. Still other embodiments include integration of a maltogenic alpha amylase from Lactobacillus plantarum S21 (SEQ ID NO: 1), glucoamylase from Saccharomycopsis fibuligera (SEQ ID NO: 2), and a glucoamylase from Penicillium oxalicum (SEQ ID NO:3) into other yeast strains important for ethanol production. In another embodiment, the maltogenic alpha amylase and the two glucoamylases are not integrated into the yeast genome, instead they are expressed and maintained on a plasmid. The plasmid may either be maintained at one copy per cell or as multiple copies per cell. This is dictated by the plasmid type. The plasmid may contain a CEN/ARS sequence allowing replication and faithful transmission to daughter cells. Furthermore, the alpha amylase and the glucoamylases may be expressed from the same plasmid or two or three separate plasmids.

A first embodiment includes a recombinant yeast strain, comprising a strain of S. cerevisiae, an exogenous MALI gene cluster, an exogenous MAL2-8c gene, and an exogenous maltogenic alpha amylase gene from Lactobacillus plantarum S21; wherein the strain of S. cerevisiae expresses the exogenous MALI gene cluster, the exogenous MAL2-8c gene, and the exogenous maltogenic alpha amylase gene from Lactobacillus plantarum S21.

A second embodiment includes the recombinant yeast strain according to the first embodiment, wherein the exogenous maltogenic alpha amylase from Lactobacillus plantarum S21 is overexpressed.

A third embodiment includes the recombinant yeast strain according to any one of the first and the second embodiments, further comprising an exogenous glucoamylase gene from Saccharaomycopsis fibuligera.

An fourth embodiment includes the recombinant yeast strain according to the third embodiment, wherein the exogenous glucoamylase gene from Saccharaomycopsis fibuligera is overexpressed.

A fifth embodiment includes the recombinant yeast strain according to any one of the third and fourth embodiments, wherein the exogenous glucoamylase gene from Saccharomycopsis fibuligera is present in more than one copy per cell

A sixth embodiment includes the recombinant yeast strain according any one of the third to the fifth embodiments, wherein the glucoamylase from Saccharomycopsis fibuligera is integrated into the genome of the strain of S. cerevisiae.

A seventh embodiment includes the recombinant yeast strain according to the sixth embodiment, wherein the exogenous glucoamylase gene from Saccharomycopsis fibuligera is integrated into the genome at different positions on more than one chromosome.

An eighth embodiment includes the recombinant yeast strain according to any one of the third to the seventh embodiments, wherein the exogenous glucoamylase gene from Saccharomycopsis fibuligera is inserted into the genome of the strain of S. cerevisiae within a region encoding the Dubious Open Reading Frame YCR022c.

A ninth embodiment includes the recombinant yeast strain according to any one of the third to the eighth embodiments, wherein the exogenous glucoamylase gene from Saccharomycopsis fibuligera is inserted into the genome of the strain of S. cerevisiae within a region encoding the Dubious Open Reading Frame YMR082c.

A tenth embodiment includes the recombinant yeast strain according to any one of the eighth and ninth embodiments, wherein the exogenous glucoamylase gene from Saccharomycopsis fibuligera is inserted into two places of the genome of the strain of S. cerevisiae, a first region encoding the Dubious Open Reading Frame YCR022c and a second region encoding the Dubious Open Reading Frame YMR082.

An eleventh embodiment includes the recombinant yeast strain according to any one of the third to the tenth embodiments, wherein the exogenous glucoamylase gene from Saccharomycopsis fibuligera comprises a sequence having at least 80% homology to SEQ ID NO: 3 and the exogenous maltogenic alpha amylase gene from Lactobacillus plantarum S21 comprises a sequence having at least 80% homology to SEQ ID NO: 1. The exogenous glucoamylase gene from Saccharomycopsis fibuligera may comprise a sequence having at least 81%, at least 82%, at least 83%, and/or at least 84% homology to SEQ ID NO: 3 and the exogenous maltogenic alpha amylase gene from Lactobacillus plantarum S21 comprises a sequence having at least 81%, at least 82%, at least 83%, and/or at least 84% homology to SEQ ID NO: 1.

A twelfth embodiment includes the recombinant yeast strain according to any one of the third to the eleventh embodiments, wherein the exogenous glucoamylase gene from Saccharomycopsis fibuligera comprises a sequence having at least 85% homology to SEQ ID NO: 3 and the exogenous maltogenic alpha amylase gene from Lactobacillus plantarum S21 comprises a sequence having at least 85% homology to SEQ ID NO: 1. The exogenous glucoamylase gene from Saccharomycopsis fibuligera may comprise a sequence having at least 86%, at least 87%, at least 88%, and/or at least 89% homology to SEQ ID NO: 3 and the exogenous maltogenic alpha amylase gene from Lactobacillus plantarum S21 comprises a sequence having at least 86%, at least 87%, at least 88%, and/or at least 89% homology to SEQ ID NO: 1.

A thirteenth embodiment includes the recombinant yeast strain according to any one of the third to the twelfth embodiments, wherein the exogenous glucoamylase gene from Saccharomycopsis fibuligera comprises a sequence having at least 90% homology to SEQ ID NO: 3 and the exogenous maltogenic alpha amylase gene from Lactobacillus plantarum S21 comprises a sequence having at least 90% homology to SEQ ID NO: 1. The exogenous glucoamylase gene from Saccharomycopsis fibuligera may comprise a sequence having at least 91%, at least 92%, at least 93%, and/or at least 94% homology to SEQ ID NO: 3 and the exogenous maltogenic alpha amylase gene from Lactobacillus plantarum S21 comprises a sequence having at least 91%, at least 92%, at least 93%, and/or at least 94% homology to SEQ ID NO: 1.

A fourteenth embodiment includes the recombinant yeast strain according to any one of the third to the thirteenth embodiments, wherein the exogenous glucoamylase gene from Saccharomycopsis fibuligera comprises a sequence having at least 95% homology to SEQ ID NO: 3 and the exogenous maltogenic alpha amylase gene from Lactobacillus plantarum S21 comprises a sequence having at least 95% homology to SEQ ID NO: 1. The exogenous glucoamylase gene from Saccharomycopsis fibuligera may comprise a sequence having at least 96%, at least 97%, at least 98%, and/or at least 99% homology to SEQ ID NO: 3 and the exogenous maltogenic alpha amylase gene from Lactobacillus plantarum S21 comprises a sequence having at least 96%, at least 97%, at least 98%, and/or at least 99% homology to SEQ ID NO: 1.

A fifteenth embodiment includes the recombinant yeast strain according to any one of the third to the fourteenth embodiments, wherein the exogenous glucoamylase gene from Saccharomycopsis fibuligera comprises a sequence having SEQ ID NO: 3 and the exogenous maltogenic alpha amylase gene from Lactobacillus plantarum S21 comprises a sequence having SEQ ID NO: 1.

A sixteenth embodiment includes the recombinant yeast strain according to any one of the first to the fifteenth embodiments, further comprising an exogenous glucoamylase from Penicillium oxalicum.

A seventeenth embodiment includes the recombinant yeast strain according the sixteenth embodiment, wherein the exogenous glucoamylase gene from Penicillium oxalicum is overexpressed.

An eighteenth embodiment includes the recombinant yeast strain according to any one of the sixteenth and the seventeenth embodiments, wherein the exogenous glucoamylase gene from Penicillium oxalicum is integrated into the genome of the strain of S. cerevisiae.

A nineteenth embodiment includes the recombinant yeast strain according to any one of the sixteenth to the eighteenth embodiments, wherein the exogenous glucoamylase gene from Penicillium oxalicum is integrated into the genome of the strain of S. cerevisiae.

A twentieth embodiment includes the recombinant yeast strain according to any one of the sixteenth to the nineteenth embodiments, wherein the exogenous glucoamylase gene from Penicillium oxalicum comprises a sequence having at least 80% homology to SEQ ID NO: 5 and the exogenous maltogenic alpha amylase gene from Lactobacillus plantarum S21 comprises a sequence having at least 80% homology to SEQ ID NO: 1. The exogenous glucoamylase gene from Penicillium oxalicum may comprise a sequence having at least 81%, at least 82%, at least 83%, and/or at least 84% homology to SEQ ID NO: 5 and the exogenous maltogenic alpha amylase gene from Lactobacillus plantarum S21 comprises a sequence having at least 81%, at least 82%, at least 83%, and/or at least 84% homology to SEQ ID NO: 1.

A twenty-first embodiment includes the recombinant yeast strain according to any one of the sixteenth to the twentieth embodiments, wherein the exogenous glucoamylase gene from Penicillium oxalicum comprises a sequence having at least 85% homology to SEQ ID NO: 5 and the exogenous maltogenic alpha amylase gene from Lactobacillus plantarum S21 comprises a sequence having at least 85% homology to SEQ ID NO: 1. The exogenous glucoamylase gene from Penicillium oxalicum may comprise a sequence having at least 86%, at least 87%, at least 88%, and/or at least 89% homology to SEQ ID NO: 5 and the exogenous maltogenic alpha amylase gene from Lactobacillus plantarum S21 comprises a sequence having at least 86%, at least 87%, at least 88%, and/or at least 89% homology to SEQ ID NO: 1.

A twenty-second embodiment includes the recombinant yeast strain according to any one of the sixteenth to the twenty-first embodiments, wherein the exogenous glucoamylase gene from Penicillium oxalicum comprises a sequence having at least 90% homology to SEQ ID NO: 5 and the exogenous maltogenic alpha amylase gene from Lactobacillus plantarum S21 comprises a sequence having at least 90% homology to SEQ ID NO: 1. The exogenous glucoamylase gene from Penicillium oxalicum may comprise a sequence having at least 91%, at least 92%, at least 93%, and/or at least 94% homology to SEQ ID NO: 5 and the exogenous maltogenic alpha amylase gene from Lactobacillus plantarum S21 comprises a sequence having at least 91%, at least 92%, at least 93%, and/or at least 94% homology to SEQ ID NO: 1.

A twenty-third embodiment includes the recombinant yeast strain according to any one of the sixteenth to the twenty-second embodiments, wherein the exogenous glucoamylase gene from Penicillium oxalicum comprises a sequence having at least 95% homology to SEQ ID NO: 5 and the exogenous maltogenic alpha amylase gene from Lactobacillus plantarum S21 comprises a sequence having at least 95% homology to SEQ ID NO: 1. The exogenous glucoamylase gene from Penicillium oxalicum may comprise a sequence having at least 96%, at least 97%, at least 98%, and/or at least 99% homology to SEQ ID NO: 5 and the exogenous maltogenic alpha amylase gene from Lactobacillus plantarum S21 comprises a sequence having at least 96%, at least 97%, at least 98%, and/or at least 99% homology to SEQ ID NO: 1.

A twenty-fourth embodiment includes the recombinant yeast strain according to any one of the sixteenth to the twenty-third embodiments, wherein the exogenous glucoamylase gene from Penicillium oxalicum comprises a sequence having SEQ ID NO: 5 and the exogenous maltogenic alpha amylase gene from Lactobacillus plantarum S21 comprises a sequence having SEQ ID NO: 1.

A twenty-fifth embodiment includes the recombinant yeast strain according to any one of the first to the twenty-fourth embodiments, wherein the exogenous maltogenic alpha amylase from Lactobacillus plantarum S21 gene is integrated into the genome of the strain of S. cerevisiae.

A twenty-sixth embodiment includes the recombinant yeast strain according to any one of the first to the twenty-fifth embodiments, wherein the exogenous maltogenic alpha amylase from Lactobacillus plantarum S21 is inserted into the genome of the strain of S. cerevisiae within a region encoding the Dubious Open Reading Frame YCR022c.

A twenty-seventh embodiment includes the recombinant yeast strain according to any one of the first to the twenty-sixth embodiments, wherein the strain of S. cerevisiae is haploid, diploid, or has a ploidy number greater than two.

A twenty-eighth embodiment includes the recombinant yeast strain according to any one of the first to the twenty-seventh embodiments, wherein the recombinant yeast strain is made using genetic engineering or wherein the recombinant yeast strain is genetically modified.

A twenty-ninth embodiment includes any one of the first to the twenty-eighth embodiments, wherein the recombinant yeast strain is capable of fermenting maltose as well as disaccharides and trisaccharides comprised of glucose while simultaneously improving the efficiency and speed of glucose fermentation and eliminating the requirement for supplemental glucoamylase.

A thirtieth embodiment includes a vector comprising a maltogenic alpha amylase gene from Lactobacillus plantarum S21 that comprises a sequence having at least 80%, at least 85%, at least 90%, at least 95%, at least 98%, or at least 100% homology or identity to SEQ ID NO: 1. The maltogenic alpha amylase gene from Lactobacillus plantarum S21 may comprise a sequence having at least 81%, at least 82%, at least 83%, at least 84%, at least 86%, at least 87%, at least 88%, at least 89%, at least 91%, at least 92%, at least 93%, at least 94%, at least 96%, at least 97%, at least 98%, and/or at least 99% homology to SEQ ID NO: 1.

A thirty-first embodiment includes the vector according to the thirtieth embodiment, further comprising a glucoamylase gene from Saccharomycopsis fibuligera that comprises a sequence having at least 80%, at least 85%, at least 90%, at least 95%, at least 98%, or at least 100% homology or identity to SEQ ID NO: 3. The glucoamylase gene from Saccharomycopsis fibuligera may comprise a sequence having at least 81%, at least 82%, at least 83%, at least 84%, at least 86%, at least 87%, at least 88%, at least 89%, at least 91%, at least 92%, at least 93%, at least 94%, at least 96%, at least 97%, at least 98%, and/or at least 99% homology to SEQ ID NO: 3.

A thirty-second embodiment includes the vector according to the thirty-first embodiment, further comprising a glucoamylase gene from Penicillium oxalicum that comprises a sequence having at least 80%, at least 85%, at least 90%, at least 95%, at least 98%, or at least 100% homology or identity to SEQ ID NO: 5. The glucoamylase gene from Penicillium oxalicum may comprise a sequence having at least 81%, at least 82%, at least 83%, at least 84%, at least 86%, at least 87%, at least 88%, at least 89%, at least 91%, at least 92%, at least 93%, at least 94%, at least 96%, at least 97%, at least 98%, and/or at least 99% homology to SEQ ID NO: 5.

A thirty-third embodiment includes the vector according to any one of the thirtieth to the thirty-second embodiments, wherein the maltogenic alpha amylase gene from Lactobacillus plantarum S21 and/or the glucoamylase gene from Saccharomycopsis fibuligera and/or the glucoamylase gene from Penicillium oxalicum are maintained and expressed in a haploid, diploid, or polyploid of a strain of S. cerevisiae.

A thirty-fourth embodiment includes the vector according to any one of the thirtieth to the thirty-third embodiments, wherein the vector is expressed in the strain of S. cerevisiae as a single copy or multiple copies. Consistent with these embodiments, the vector and/or plasmid may either be maintained at one copy per cell or as multiple copies per cell.

A thirty-fifth embodiment includes a vector comprising a glucoamylase gene from Penicillium oxalicum that comprises a sequence having at least 80%, at least 85%, at least 90%, at least 95%, at least 98%, or at least 100% homology or identity to SEQ ID NO: 5. The glucoamylase gene from Penicillium oxalicum may comprise a sequence having at least 81%, at least 82%, at least 83%, at least 84%, at least 86%, at least 87%, at least 88%, at least 89%, at least 91%, at least 92%, at least 93%, at least 94%, at least 96%, at least 97%, at least 98%, and/or at least 99% homology to SEQ ID NO: 5.

A thirty-sixth embodiment includes the vector according to the thirty-fifth embodiment, wherein the glucoamylase gene from Penicillium oxalicum is maintained and expressed in a haploid, diploid, or polyploid of a strain of S. cerevisiae.

A thirty-seventh embodiment includes the vector according to the thirty-sixth embodiment, wherein the vector is expressed in the strain of S. cerevisiae as a single copy or multiple copies.

A thirty-eighth embodiment includes a vector comprising a glucoamylase gene from Saccharomycopsis fibuligera having at least 80%, at least 85%, at least 90%, at least 95%, at least 98%, or at least 100% homology or identity to SEQ ID NO: 3. The glucoamylase gene from Saccharomycopsis fibuligera may comprise a sequence having at least 81%, at least 82%, at least 83%, at least 84%, at least 86%, at least 87%, at least 88%, at least 89%, at least 91%, at least 92%, at least 93%, at least 94%, at least 96%, at least 97%, at least 98%, and/or at least 99% homology to SEQ ID NO: 3.

A thirty-ninth embodiment includes the vector according to the thirty-eighth embodiment, wherein the glucoamylase gene from Saccharomycopsis fibuligera is maintained and expressed in a haploid, diploid, or polyploid of a strain of S. cerevisiae.

A fortieth embodiment includes the vector according to the thirty-ninth embodiment, wherein the vector is expressed in the strain of S. cerevisiae as a single copy or multiple copies.

A forty-first embodiment includes a method of producing a recombinant yeast strain, comprising: integrating an exogenous alpha amylase gene from Lactobacillus plantarum S21 having at least 80% homogeny to SEQ ID NO: 1 and/or an exogenous glucoamylase gene from Saccharomycopsis fibuligera having at least 80% homogeny to SEQ ID NO: 3 and/or an exogenous glucoamylase gene from Penicillium oxalicum having at least 80% homogeny to SEQ ID NO: 5 into the genome of a strain of S. cerevisiae.

### BRIEF DESCRIPTION OF THE FIGURES

FIG. 1. is a schematic drawing illustrating composition of the pDNLS3-HGHP plasmid.

FIG. 2. is a schematic drawing illustrating the actual arrangement of the HGHP gene cassette inserted into NLS3.

FIG. 3. is a schematic drawing illustrating composition of the pDNLS7-HMHG plasmid.

FIG. 4. is a schematic drawing illustrating the actual arrangement of the HMHG gene cassette inserted into NLS7.

FIG. 5. is a schematic drawing illustrating details of the genomic features and gene expression profiles around dubious ORF YMR082C, termed “Neutral Landing Site #3”, the site of HGHP. YMR082C is a dubious Open reading frame whose transcript does not code for a functional protein.

FIG. 6. is a schematic drawing illustrating details of the genomic features and gene expression profiles around dubious ORF YCR022C, termed “Neutral Landing Site #7”, the site of HMHG. YMR022C is a dubious Open reading frame whose transcript does not code for a functional protein.

FIG. 7A is a graph illustrating the changes in ethanol levels from the leading GMO yeast and the F20 yeast strain under standard fermentation conditions when maltose and glucose corn mash is treated with either a none or a 0.02% solution of Ultra F glucoamylase (Novozymes).

FIG. 7B is a graph illustrating the changes in total sugar levels from the leading GMO yeast strain compared to the F20 yeast strain under standard fermentation conditions when corn mash is treated with either a none or a 0.02% (w/w) solution of Ultra F glucoamylase (Novozymes).

FIG. 7C is a graph illustrating the changes in maltose levels from the leading GMO yeast strain compared to the F20 yeast strain under standard fermentation conditions when corn mash is treated with a 0.02% (w/w) solution of Ultra F glucoamylase (Novozymes).

FIG. 7D is a graph illustrating the changes in DP3 levels from the leading GMO yeast strain compared to the F20 yeast strain under standard fermentation conditions when corn mash is treated with a 0.02% (w/w) solution of Ultra F glucoamylase (Novozymes).

### SEQUENCE LISTING

SEQ ID NO: 1. CODON OPTIMIZED MALTOGENIC ALPHA AMYLASE (MALPS21) FROM Lactobacillus plantarum S21

| | |
|---|---|
| FEATURES | Location/Qualifiers |
| CDS | 1..2625/“MALPS21″ |

| ORIGIN | |
|---|---|
| 1 | gattcataca ccacctcaac agacgattcg tctaatgaca ctgccgacag tgtctctgat |
| 61 | ggtgtgattt tacacgcttg gtgttggtct ttcaacacaa tcaagaacaa tttgaagcaa |
| 121 | attcacgatg caggttacac tgccgttcaa acctcccctg tcaatgaagt caaagttggt |
| 181 | aattctgcta gtaagtcttt gaacaactgg tactggttat accaaccaac aaagtactcg |
| 241 | attggtaact attacttagg taccgaagct gaattcaagt ccatgtgtgc agctgccaag |
| 301 | gagtacaaca tcagaattat tgttgatgct accttgaatg acaccacaag tgactactca |
| 361 | gctatttcgg atgaaatcaa atccattagt aattggactc atggcaatac acagatatcc |
| 421 | aactggtcag acagggagga tgtcacccaa aactctctcc ttggtttgta tgattggaac |
| 481 | actcaaaatt cccaagtcca aacataccta aagaactact tggaacgtct aatatcagat |
| 541 | ggggcaagcg gttttcgtta cgatgcagcc aaacatatcg aattgccatc acaatacgac |
| 601 | ggttcatatg gttccaattt ttggccaaat atcactgaca atggtagtga attccaatat |
| 661 | ggcgaagttt tgcaagattc tatttccaaa gaatccgatt acgctaatta catgtcagta |
| 721 | acagcctcta attatggtaa tactattaga aatgccctga aaaacagaga tttcactgct |
| 781 | agcacattac aaaatttcaa tatttctgtc cccgctagca agttggttac ttgggttgaa |
| 841 | tctcatgaca actatgcaaa cgatgaccaa gtttctacct ggatgaatag ttccgatatt |
| 901 | aaactaggtt gggccgtagt ggcctcaaga tctggaagtg ttccattatt tttcgacaga |
| 961 | ccagttgacg gtggtaatgg tacccgtttt cctggatcta gcgaaattgg tgacgccggt |
| 1021 | tcttcgcttt attatgacaa ggctgttgtg gcggttaaca agttccacaa cgccatggct |
| 1081 | ggtcaatctg aatacatttc aaacccaaac ggtaacacca aaatttttga aaacgaaaga |
| 1141 | ggttctaagg gtgtcgtttt cgctaatgct tcggatggca gctattctct atctgttaag |
| 1201 | acatctcttg ctgacggtac ctacgaaaat aaggccggaa gtgacgagtt cactgttaaa |
| 1261 | aacggttatt tgacaggtac tatccaaggt agagaagtag tcgtattata tggcgatcca |
| 1321 | acttcaagct cgtcctcgtc taccactact gaaactaaga aggtgtattt tgaaaaacca |
| 1381 | tcctcctggg gttccacagt ctatgcctat gtctacaaca aaaacactaa taaggctata |
| 1441 | accagcgcat ggccaggtaa agagatgact gctttaggta atgatgagta taaattagac |
| 1501 | ctggatacag atgaagatga ttccgacttg gcagtaattt tcaccgatgg gaccaaccaa |
| 1561 | actcctgcag ccaacaaggc tgggttcacc ttcacagcag acgcgacgta cgatcagaac |
| 1621 | ggtgttgtta agacctctga ctcatcttcg tcgtcctcca ctaccaccga aacaaaaaaa |
| 1681 | gtgtattttg aaaagccttc atcttggggg tccactgtct acgcctacgt ttataataaa |
| 1741 | aacacgaaca aagctatcac cagtgcttgg cccggtaagg aaatgaccgc tcttggaaat |
| 1801 | gacgaatata aattggattt ggatactgat gaagatgata gtgatctagc tgttatcttt |
| 1861 | actgatggta caaaccaaac gccggcagct aacaaggcag gtttcacttt taccgctgat |
| 1921 | gccacttatg atcaaaacgg tgtggttaag acatctgaca gttcttcatc atcttccagt |
| 1981 | acaactacgg aaactaagaa agtttacttc gaaaagccat cttcgtgggg ctctacggtt |
| 2041 | tacgcttatg tttataacaa gaatacaaat aaagcaatta cttccgcttg gcctggtaag |
| 2101 | gaaatgactg cgttaggcaa cgacgaatac aagttagatt tagataccga tgaagatgat |
| 2161 | agtgatttgg ctgtgatctt cactgatgga accaaccaga ctccagctgc taacaaagca |
| 2221 | ggctttacct ttactgctga tgccacttat gaccagaatg gtgttgtcaa gacctccgat |
| 2281 | agctcctctt cctcgtcaac tactacagaa acgaagaagg tttactttga gaagccaagt |
| 2341 | agttggggtt ctacagttta tgcttacgta tacaataaaa atactaataa agcgatcact |
| 2401 | agcgcctggc caggtaaaga aatgacagct ttgggcaatg acgaatacaa attggacctt |
| 2461 | gacactgacg aggacgactc cgatttggct gttatattta ccgatggtac taatcaaacg |
| 2521 | cctgctgcaa ataaagctgg tttcacattt accgccgatg ctacttacga tcagaacggt |
| 2581 | gtcgtcaaaa catctgattc ttcgtccacc tcttctacat cataa |

SEQ ID NO: 2. PREDICTED PROTEIN PRODUCT OF CODON OPTIMIZED Lactobacillus plantarum S21 (MALPS21) (SEQUENCE NUMBER 1)

| | |
|---|---|
| FEATURES | Location/Qualifiers |
| CDS | 1..>874/“MALPS21″ |

| ORIGIN | |
|---|---|
| 1 | dsyttstdds sndtadsvsd gvilhawcws fntiknnlkq ihdagytavq tspvnevkvg |
| 61 | nsaskslnnw ywlyqptkys ignyylgtea efksmcaaak eyniriivda tlndttsdys |
| 121 | aisdeiksis nwthgntqis nwsdredvtq nsllglydwn tqnsqvqtyl knylerlisd |
| 181 | gasgfrydaa khielpsqyd gsygsnfwpn itdngsefqy gevlqdsisk esdyanymsv |
| 241 | tasnygntir nalknrdfta stlqnfnisv pasklvtwve shdnyanddq vstwmnssdi |
| 301 | klgwavvasr sgsvplffdr pvdggngtrf pgsseigdag sslyydkavv avnkfhnama |
| 361 | gqseyisnpn gntkifener gskgvvfana sdgsyslsvk tsladgtyen kagsdeftvk |
| 421 | ngyltgtiqg revvvlygdp tssssssttt etkkvyfekp sswgstvyay vynkntnkai |
| 481 | tsawpgkemt algndeykld ldtdeddsdl aviftdgtnq tpaankagft ftadatydqn |
| 541 | gvvktsdsss ssstttetkk vyfekpsswg stvyayvynk ntnkaitsaw pgkemtalgn |
| 601 | deykldldtd eddsdlavif tdgtnqtpaa nkagftftad atydqngvvk tsdsssssss |
| 661 | tttetkkvyf ekpsswgstv yayvynkntn kaitsawpgk emtalgndey kldldtdedd |
| 721 | sdlaviftdg tnqtpaanka gftftadaty dqngvvktsd ssssssttte tkkvyfekps |
| 781 | swgstvyayv ynkntnkait sawpgkemta lgndeykldl dtdeddsdla viftdgtnqt |
| 841 | paankagftf tadatydqng vvktsdssst ssts |

SEQ ID NO: 3. CODON OPTIMIZED GLUCOAMYLASE (GLM) FROM Saccharomycopsis fibuligera

| | |
|---|---|
| FEATURES | Location/Qualifiers |
| CDS | 1..1470/“Glm″ |

| ORIGIN | |
|---|---|
| 1 | aatacaggtc atttccaagc ctactctggt tacacagttg ctcgttccaa cttcacccaa |
| 61 | tggattcacg aacaacctgc cgtgtcatgg tattatttgc ttcagaatat tgactaccca |
| 121 | gaaggccagt tcaaatcggc caagcctggt gttgttgtgg ccagcccatc tacttcagag |
| 181 | ccagattact tttaccaatg gactagagat actgcaatta ctttcttgag tttgattgct |
| 241 | gaagttgaag accattcttt ttcaaacact actttggcta aggtcgttga atactacatt |
| 301 | tcaaatacat acaccttaca aagagtatcg aacccatcag gtaactttga cagcccaaac |
| 361 | catgatggtt taggtgaacc aaagtttaat gtggatgata ccgcatatac tgcttcttgg |
| 421 | ggtcgtcctc aaaatgacgg tccagctttg agagcttatg ctatttctag gtatctgaat |
| 481 | gccgtcgcca aacacaacaa cggtaagttg ctgctcgcgg gccaaaacgg tataccgtat |
| 541 | tcttctgcct ctgatatcta ctggaaaatt attaaacctg atttacaaca tgtttccacc |
| 601 | cattggtcta cctccggatt tgatttgtgg gaagagaacc aaggtactca cttcttcacg |
| 661 | gcactagtgc agttgaaagc tctatcttat ggtattcctt tgtccaagac ttataatgat |
| 721 | ccagggttta cctcgtggtt ggaaaagcaa aaggatgctt taaattccta cataaattct |
| 781 | tccggtttcg ttaattcagg caaaaagcac attgtcgaat ctccacaact tagttctaga |
| 841 | ggtggtttgg actcagctac ctatatcgcc gctctaatca cccacgatat tggtgacgat |
| 901 | gacacctaca ctccattcaa tgtcgacaac agctatgtct taaacagttt atattactta |
| 961 | ttggttgata acaagaatcg ttataaaatc aacggaaact acaaggctgg tgctgctgtt |
| 1021 | ggtagatatc ctgaagatgt ttacaatggt gtcggaactt ctgaaggtaa tccatggcaa |
| 1081 | ttggccactg cctacgctgg tcaaactttt tatacattag cttacaactc cttgaagaac |
| 1141 | aagaaaaatt tagtaattga aaaattgaac tatgacttgt acaactcttt catagctgat |
| 1201 | ctatcgaaga tcgatagttc ctatgcaagt aaggactctt taacacttac ttacggttcc |
| 1261 | gacaattaca aaaacgttat caaatccttg ctacaatttg gtgattcctt tttaaaggtt |
| 1321 | ttgttggatc atattgatga taatggtcaa ttaactgaag aaattaacag atacactggt |
| 1381 | tttcaagctg gcgccgtatc attgacatgg tcctccggtt ctttgttgtc tgctaatagg |
| 1441 | gcaagaaaca aattaatcga gctattataa |

SEQ ID NO: 4. PREDICTED PROTEIN PRODUCT OF CODON OPTIMIZED Saccharomycopsis fibuligera GLUCOAMYLASE (GLM) (SEQUENCE NUMBERS 3)

| | |
|---|---|
| FEATURES | Location/Qualifiers |
| CDS | 1..>489/″Glm″ |

| ORIGIN | |
|---|---|
| 1 | ntghfqaysg ytvarsnftq wiheqpavsw yyllqnidyp egqfksakpg vvvaspstse |
| 61 | pdyfyqwtrd taitflslia evedhsfsnt tlakvveyyi sntytlqrvs npsgnfdspn |
| 121 | hdglgepkfn vddtaytasw grpqndgpal rayaisryln avakhnngkl llagqngipy |
| 181 | ssasdiywki ikpdlqhvst hwstsgfdlw eenqgthfft alvqlkalsy giplsktynd |
| 241 | pgftswlekq kdalnsyins sgfvnsgkkh ivespqlssr ggldsatyia alithdigdd |
| 301 | dtytpfnvdn syvlnslyyl lvdnknryki ngnykagaav grypedvyng vgtsegnpwq |
| 361 | latayagqtf ytlaynslkn kknlviekln ydlynsfiad lskidssyas kdsltltygs |
| 421 | dnyknviksl lqfgdsflkv lldhiddngq lteeinrytg fqagavsltw ssgsllsanr |
| 481 | arnkliell |

SEQ ID NO: 5. CODON OPTIMIZED GLUCOAMYLASE (GLM) FROM Penicillium oxalicum

| | |
|---|---|
| FEATURES | Location/Qualifiers |
| CDS | 1..1851/″PoGA″ |

| ORIGIN | |
|---|---|
| 1 | gccccacaat tgtcccccag ggctacttct ctagattcct ggttatccag cgaaactact |
| 61 | ttttctttga acggtattct cgccaacatc ggttcttctg gtgcttactc taagtctgct |
| 121 | gcctctggtg ccgtcatcgc ttccccttct actagcaacc ccgattacta ttatacctgg |
| 181 | accagagacg cagcgttaac tttgaaagcc ttagttgata ttttccgtaa tggcaatttg |
| 241 | ggtctacaaa ccgttatcga acaatatgtt aatgcacagg ctaaattgca aactgtctct |
| 301 | aatccttccg gaggtttgtc cgacggtgca ggtttgggag aacctaagtt caatgttgac |
| 361 | ttgtctgctt tcactggtgc ttggggtaga ccacaaagag atggcccggc tctacgggct |
| 421 | atagcactaa tcgatttcgg caattggctg atagataacg gatataaatc ttacgcggtg |
| 481 | aacaacgttt ggccaatcgt aaggaacgat ttggcctatg ttgcccagta ctggtcacag |
| 541 | tccggcttcg acctatggga agaagtgaat tctatgtctt tctttacagt tgctaaccaa |
| 601 | catcgttcat tagtcgaagg atcagctttc gcatctcgtg tcggtgccag ctgttctggt |
| 661 | tgtgactctc aagctcctca gattttgtgt tacatgcaat ctttttggac tgggagttat |
| 721 | attaatgcca atacgggtgg tggtagatcc ggtaaagatt ctaacactat tttagcctcg |
| 781 | atacatactt ttgatcctgc tgcttcttgt gatgacgtta ccttccaacc atgctcaagt |
| 841 | agagctttgg ctaaccacaa ggtctatacc gattctttca gatccgttta cgcgttaaac |
| 901 | tccggtatag cccaaggtaa ggccgtttct gtaggtcgtt acccagaaga tagttactac |
| 961 | ggtggcaacc catggttttt atcaaactta gcagctgctg agcaacttta tgatgctatc |
| 1021 | taccaatgga acaagattgg ttccatcact atcacctcga cctcgcttgc atttttcaag |
| 1081 | gatgtttatc cgtctgccgc taccggtacc tatgcttctg ggtccacaac ctttaatgct |
| 1141 | attatttctg cagtaaagac atatgctgac ggctatgtca gtattgttca atcccactcc |
| 1201 | tatgcgaatg gttcgttgtc agaacaattc gacagaacca ctggtttgtc catcagtgct |
| 1261 | cgcgatttaa catggtctta tgcggcgctg ttgactgcaa atgacagaag aaatggcgtt |
| 1321 | gtccctccat cgtggggcgc aagttccgct aattcgatac ctggttcatg cagcatgggt |
| 1381 | tctgccacag gttcctacgc tactccatct gttggttcat ggccagcaac acttacttca |
| 1441 | ggtacagctg caccttccag tacatcaact actaccaagg ctccaactac caccacggcc |
| 1501 | accacaacaa cttccgccgg ttcctgtact acaccaaccg cagtggctgt tactttcgat |
| 1561 | gaaattgcta cgacgacatt tggtgaaaac gtctacttgg taggaagcat tagccaatta |
| 1621 | ggtaactgga atacagccaa cggtatccca ctgtctgctt caaagtacac ctcttcaaat |
| 1681 | ccattatggt acgccactgt gaacttgccc gctggcacta cttttcaata caaatatttt |
| 1741 | agaaaggaat ctgatggttc catcaaatgg gagtcagacc caaacagatc ttacactgtt |
| 1801 | ccagccaaat gtggtactac tacagccaca gaaaatgata cttggagata a |

SEQ ID NO: 6. PREDICTED PROTEIN PRODUCT OF CODON OPTIMIZED Penicillium oxalicum (PoGA) (SEQUENCE NUMBER 5)

| | |
|---|---|
| FEATURES | Location/Qualifiers |
| CDS | 1..>616/″PoGA″ |

| ORIGIN | |
|---|---|
| 1 | apqlsprats ldswlssett fslngilani gssgaysksa asgaviasps tsnpdyyytw |
| 61 | trdaaltlka lvdifrngnl glqtvieqyv naqaklqtvs npsgglsdga glgepkfnvd |
| 121 | lsaftgawgr pqrdgpalra ialidfgnwl idngyksyav nnvwpivrnd layvagywsq |
| 181 | sgfdlweevn smsfftvanq hrslvegsaf asrvgascsg cdsqapqilc ymqsfwtgsy |
| 241 | inantgggrs gkdsntilas ihtfdpaasc ddvtfqpcss ralanhkvyt dsfrsvyaln |
| 301 | sgiaqgkavs vgrypedsyy ggnpwflsnl aaaeqlydai yqwnkigsit itstslaffk |
| 361 | dvypsaatgt yasgsttfna iisavktyad gyvsivqshs yangslseqf drttglsisa |
| 421 | rdltwsyaal ltandrrngv vppswgassa nsipgscsmg satgsyatps vgswpatlts |
| 481 | gtaapsstst ttkaptttta ttttsagsct tptavavtfd eiatttfgen vylvgsisql |
| 541 | gnwntangip lsaskytssn plwyatvnlp agttfqykyf rkesdgsikw esdpnrsytv |
| 601 | pakcgtttat endtwr |

SEQ ID NO: 7. HGHP genomic insertion sequence at NLS3

| | |
|---|---|
| FEATURES | Location/Qualifiers |
| misc_feature | <1..286/“UPS_NLS3” |
| terminator | 295..484/“Terminator CYC1″ |
| promoter | 495..1221/“HOR7 promoter″ |
| sig_peptide | 1222..1299/“GLM signal peptide″ |
| CDS | 1300..2769/“GLM″ |
| terminator | 2770..3197/“PGK1 terminator″ |
| promoter | 3198..3924/“HOR7 promoter″ |
| sig_peptide | 3925..400/“GLM signal peptide″ |
| CDS | 4003..5472/“GLM″ |
| terminator | 5473..5900/“PGK1 terminator″ |
| promoter | 5901..662/HOR7 promoter″ |
| sig_peptide | 6628..6705/“GLM signal peptide″ |
| CDS | 6706..8175/“Glm″ |
| terminator | 8176..8603/“Terminator PGK1″ |
| misc_feature | 8604..9330/“Promoter HOR7″ |
| sig_peptide | 9331..9408/“GLM signal peptide″ |
| CDS | 9409..11259/“PoGA″ |
| terminator | 11268..11462/“Terminator ADH1″ |
| misc_feature | 11471..>11648/“DWS_NLS3″ |

| ORIGIN | |
|---|---|
| 1 | ccagtttttc catgctgggt ttcttttcgt taatagtggt gggtaaaaga aaacgtacga |
| 61 | ataaaatgct gaatgtagaa tatcctgtag gctcattaat acacagtaga acgcagaccc |
| 121 | attcgagggg ctcattggaa acacgtagtc gacattagtt ctagataatc cgcttgatgg |
| 181 | gccacatatg gtaatggctt ctcgaagcag atgttacgag ccgccagaac gaggcggtgg |
| 241 | catctgcctc gcgctgtttt ctagcggcag agaaaacccg tggatagttt aaaccttcga |
| 301 | gcgtcccaaa accttctcaa gcaaggtttt cagtataatg ttacatgcgt acacgcgttt |
| 361 | gtacagaaaa aaaagaaaaa tttgaaatat aaataacgtt cttaatacta acataactat |
| 421 | aaaaaaataa atagggacct agacttcagg ttgtctaact ccttcctttt cggttagagc |
| 481 | ggatatttcg aaatctttcg attagcacgc acacacatca catagactgc gtcataaaaa |
| 541 | tacactacgg aaaaaccata aagagcaaag cgatacctac ttggaaggaa aaggagcacg |
| 601 | cttgtaaggg ggatgggggc taagaagtca ttcactttct tttcccttcg cggtccggac |
| 661 | ccgggacccc tcctctcccc gcacaatttc ttcctttcat atcttccttt tattcctatc |
| 721 | ccgttgaagc aaccgcacta tgactaaatg gtgctggaca tctccatggc tgtgacttgt |
| 781 | gtgtatctca cagtggtaac ggcaccgtgg ctcggaaacg gttccttcgt gacaattcta |
| 841 | gaacaggggc tacagtctcg ataatagaat aataagcgca tttttgttag cgccgccgcg |
| 901 | gcgcccgttt cccaataggg aggcgcagtt tatcggcgga gctttacttc ttcctatttg |
| 961 | ggtaagcccc tttctgtttt cggccagtgg ttgctgcagg ctgcgccgga gaacatagtg |
| 1021 | ataagggatg taactttcga tgagagaatt agcaagcgga aaaaaaacta tggctagctg |
| 1081 | ggagttgttt ttcaatcata taaaagggag aaattgttgc tcactatgtg acagtttctg |
| 1141 | ggacgtctta acttttattg cagaggacta tcaaatcata cagatattgt caaaaaaaaa |
| 1201 | aaaaaagact aataataaaa aatgatcaga ttgactgtct tcttaaccgc tgttttcgca |
| 1261 | gctgtcgcat cttgtgttcc cgttgagctt gacaagagaa atacaggtca tttccaagcc |
| 1321 | tactctggtt acacagttgc tcgttccaac ttcacccaat ggattcacga acaacctgcc |
| 1381 | gtgtcatggt attatttgct tcagaatatt gactacccag aaggccagtt caaatcggcc |
| 1441 | aagcctggtg ttgttgtggc cagcccatct acttcagagc cagattactt ttaccaatgg |
| 1501 | actagagata ctgcaattac tttcttgagt ttgattgctg aagttgaaga ccattctttt |
| 1561 | tcaaacacta ctttggctaa ggtcgttgaa tactacattt caaatacata caccttacaa |
| 1621 | agagtatcga acccatcagg taactttgac agcccaaacc atgatggttt aggtgaacca |
| 1681 | aagtttaatg tggatgatac cgcatatact gcttcttggg gtcgtcctca aaatgacggt |
| 1741 | ccagctttga gagcttatgc tatttctagg tatctgaatg ccgtcgccaa acacaacaac |
| 1801 | ggtaagttgc tgctcgcggg ccaaaacggt ataccgtatt cttctgcctc tgatatctac |
| 1861 | tggaaaatta ttaaacctga tttacaacat gtttccaccc attggtctac ctccggattt |
| 1921 | gatttgtggg aagagaacca aggtactcac ttcttcacgg cactagtgca gttgaaagct |
| 1981 | ctatcttatg gtattccttt gtccaagact tataatgatc cagggtttac ctcgtggttg |
| 2041 | gaaaagcaaa aggatgcttt aaattcctac ataaattctt ccggtttcgt taattcaggc |
| 2101 | aaaaagcaca ttgtcgaatc tccacaactt agttctagag gtggtttgga ctcagctacc |
| 2161 | tatatcgccg ctctaatcac ccacgatatt ggtgacgatg acacctacac tccattcaat |
| 2221 | gtcgacaaca gctatgtctt aaacagttta tattacttat tggttgataa caagaatcgt |
| 2281 | tataaaatca acggaaacta caaggctggt gctgctgttg gtagatatcc tgaagatgtt |
| 2341 | tacaatggtg tcggaacttc tgaaggtaat ccatggcaat tggccactgc ctacgctggt |
| 2401 | caaacttttt atacattagc ttacaactcc ttgaagaaca agaaaaattt agtaattgaa |
| 2461 | aaattgaact atgacttgta caactctttc atagctgatc tatcgaagat cgatagttcc |
| 2521 | tatgcaagta aggactcttt aacacttact tacggttccg acaattacaa aaacgttatc |
| 2581 | aaatccttgc tacaatttgg tgattccttt ttaaaggttt tgttggatca tattgatgat |
| 2641 | aatggtcaat taactgaaga aattaacaga tacactggtt ttcaagctgg cgccgtatca |
| 2701 | ttgacatggt cctccggttc tttgttgtct gctaataggg caagaaacaa attaatcgag |
| 2761 | ctattataaa ttgaattgaa ttgaaatcga tagatcaatt tttttctttt ctctttcccc |
| 2821 | atcctttacg ctaaaataat agtttatttt attttttgaa tattttttat ttatatacgt |
| 2881 | atatatagac tattatttat cttttaatga ttattaagat ttttattaaa aaaaaattcg |
| 2941 | ctcctctttt aatgccttta tccagttttt ttttcccatt cgatatttct atgttcgggt |
| 3001 | tcagcgtatt ttaagtttaa taactcgaaa attctgcgtt cgttaaagct ttcgagaagg |
| 3061 | atattatttc gaaataaacc gtgttgtgta agcttgaagc ctttttgcgc tgccaatatt |
| 3121 | cttatccatc tattgtactc tttagatcca gtatagtgta ttcttcctgc tccaagttca |
| 3181 | tcccacttgc aacaaaactt tcgattagca cgcacacaca tcacatagac tgcgtcataa |
| 3241 | aaatacacta cggaaaaacc ataaagagca aagcgatacc tacttggaag gaaaaggagc |
| 3301 | acgcttgtaa gggggatggg ggctaagaag tcattcactt tcttttccct tcgcggtccg |
| 3361 | gacccgggac ccctcctctc cccgcacaat ttcttccttt catatcttcc ttttattcct |
| 3421 | atcccgttga agcaaccgca ctatgactaa atggtgctgg acatctccat ggctgtgact |
| 3481 | tgtgtgtatc tcacagtggt aacggcaccg tggctcggaa acggttcctt cgtgacaatt |
| 3541 | ctagaacagg ggctacagtc tcgataatag aataataagc gcatttttgt tagcgccgcc |
| 3601 | gcggcgcccg tttcccaata gggaggcgca gtttatcggc ggagctttac ttcttcctat |
| 3661 | ttgggtaagc ccctttctgt tttcggccag tggttgctgc aggctgcgcc ggagaacata |
| 3721 | gtgataaggg atgtaacttt cgatgagaga attagcaagc ggaaaaaaaa ctatggctag |
| 3781 | ctgggagttg tttttcaatc atataaaagg gagaaattgt tgctcactat gtgacagttt |
| 3841 | ctgggacgtc ttaactttta ttgcagagga ctatcaaatc atacagatat tgtcaaaaaa |
| 3901 | aaaaaaaaag actaataata aaaaatgatc agattgactg tcttcttaac cgctgttttc |
| 3961 | gcagctgtcg catcttgtgt tcccgttgag cttgacaaga gaaatacagg tcatttccaa |
| 4021 | gcctactctg gttacacagt tgctcgttcc aacttcaccc aatggattca cgaacaacct |
| 4081 | gccgtgtcat ggtattattt gcttcagaat attgactacc cagaaggcca gttcaaatcg |
| 4141 | gccaagcctg gtgttgttgt ggccagccca tctacttcag agccagatta cttttaccaa |
| 4201 | tggactagag atactgcaat tactttcttg agtttgattg ctgaagttga agaccattct |
| 4261 | ttttcaaaca ctactttggc taaggtcgtt gaatactaca tttcaaatac atacacctta |
| 4321 | caaagagtat cgaacccatc aggtaacttt gacagcccaa accatgatgg tttaggtgaa |
| 4381 | ccaaagttta atgtggatga taccgcatat actgcttctt ggggtcgtcc tcaaaatgac |
| 4441 | ggtccagctt tgagagctta tgctatttct aggtatctga atgccgtcgc caaacacaac |
| 4501 | aacggtaagt tgctgctcgc gggccaaaac ggtataccgt attcttctgc ctctgatatc |
| 4561 | tactggaaaa ttattaaacc tgatttacaa catgtttcca cccattggtc tacctccgga |
| 4621 | tttgatttgt gggaagagaa ccaaggtact cacttcttca cggcactagt gcagttgaaa |
| 4681 | gctctatctt atggtattcc tttgtccaag acttataatg atccagggtt tacctcgtgg |
| 4741 | ttggaaaagc aaaaggatgc tttaaattcc tacataaatt cttccggttt cgttaattca |
| 4801 | ggcaaaaagc acattgtcga atctccacaa cttagttcta gaggtggttt ggactcagct |
| 4861 | acctatatcg ccgctctaat cacccacgat attggtgacg atgacaccta cactccattc |
| 4921 | aatgtcgaca acagctatgt cttaaacagt ttatattact tattggttga taacaagaat |
| 4981 | cgttataaaa tcaacggaaa ctacaaggct ggtgctgctg ttggtagata tcctgaagat |
| 5041 | gtttacaatg gtgtcggaac ttctgaaggt aatccatggc aattggccac tgcctacgct |
| 5101 | ggtcaaactt tttatacatt agcttacaac tccttgaaga acaagaaaaa tttagtaatt |
| 5161 | gaaaaattga actatgactt gtacaactct ttcatagctg atctatcgaa gatcgatagt |
| 5221 | tcctatgcaa gtaaggactc tttaacactt acttacggtt ccgacaatta caaaaacgtt |
| 5281 | atcaaatcct tgctacaatt tggtgattcc tttttaaagg ttttgttgga tcatattgat |
| 5341 | gataatggtc aattaactga agaaattaac agatacactg gttttcaagc tggcgccgta |
| 5401 | tcattgacat ggtcctccgg ttctttgttg tctgctaata gggcaagaaa caaattaatc |
| 5461 | gagctattat aaattgaatt gaattgaaat cgatagatca atttttttct tttctctttc |
| 5521 | cccatccttt acgctaaaat aatagtttat tttatttttt gaatattttt tatttatata |
| 5581 | cgtatatata gactattatt tatcttttaa tgattattaa gatttttatt aaaaaaaaat |
| 5641 | tcgctcctct tttaatgcct ttatccagtt tttttttccc attcgatatt tctatgttcg |
| 5701 | ggttcagcgt attttaagtt taataactcg aaaattctgc gttcgttaaa gctttcgaga |
| 5761 | aggatattat ttcgaaataa accgtgttgt gtaagcttga agcctttttg cgctgccaat |
| 5821 | attcttatcc atctattgta ctctttagat ccagtatagt gtattcttcc tgctccaagt |
| 5881 | tcatcccact tgcaacaaaa ctttcgatta gcacgcacac acatcacata gactgcgtca |
| 5941 | taaaaataca ctacggaaaa accataaaga gcaaagcgat acctacttgg aaggaaaagg |
| 6001 | agcacgcttg taagggggat gggggctaag aagtcattca ctttcttttc ccttcgcggt |
| 6061 | ccggacccgg gacccctcct ctccccgcac aatttcttcc tttcatatct tccttttatt |
| 6121 | cctatcccgt tgaagcaacc gcactatgac taaatggtgc tggacatctc catggctgtg |
| 6181 | acttgtgtgt atctcacagt ggtaacggca ccgtggctcg gaaacggttc cttcgtgaca |
| 6241 | attctagaac aggggctaca gtctcgataa tagaataata agcgcatttt tgttagcgcc |
| 6301 | gccgcggcgc ccgtttccca atagggaggc gcagtttatc ggcggagctt tacttcttcc |
| 6361 | tatttgggta agcccctttc tgttttcggc cagtggttgc tgcaggctgc gccggagaac |
| 6421 | atagtgataa gggatgtaac tttcgatgag agaattagca agcggaaaaa aaactatggc |
| 6481 | tagctgggag ttgtttttca atcatataaa agggagaaat tgttgctcac tatgtgacag |
| 6541 | tttctgggac gtcttaactt ttattgcaga ggactatcaa atcatacaga tattgtcaaa |
| 6601 | aaaaaaaaaa aagactaata ataaaaaatg atcagattga ctgtcttctt aaccgctgtt |
| 6661 | ttcgcagctg tcgcatcttg tgttcccgtt gagcttgaca agagaaatac aggtcatttc |
| 6721 | caagcctact ctggttacac agttgctcgt tccaacttca cccaatggat tcacgaacaa |
| 6781 | cctgccgtgt catggtatta tttgcttcag aatattgact acccagaagg ccagttcaaa |
| 6841 | tcggccaagc ctggtgttgt tgtggccagc ccatctactt cagagccaga ttacttttac |
| 6901 | caatggacta gagatactgc aattactttc ttgagtttga ttgctgaagt tgaagaccat |
| 6961 | tctttttcaa acactacttt ggctaaggtc gttgaatact acatttcaaa tacatacacc |
| 7021 | ttacaaagag tatcgaaccc atcaggtaac tttgacagcc caaaccatga tggtttaggt |
| 7081 | gaaccaaagt ttaatgtgga tgataccgca tatactgctt cttggggtcg tcctcaaaat |
| 7141 | gacggtccag ctttgagagc ttatgctatt tctaggtatc tgaatgccgt cgccaaacac |
| 7201 | aacaacggta agttgctgct cgcgggccaa aacggtatac cgtattcttc tgcctctgat |
| 7261 | atctactgga aaattattaa acctgattta caacatgttt ccacccattg gtctacctcc |
| 7321 | ggatttgatt tgtgggaaga gaaccaaggt actcacttct tcacggcact agtgcagttg |
| 7381 | aaagctctat cttatggtat tcctttgtcc aagacttata atgatccagg gtttacctcg |
| 7441 | tggttggaaa agcaaaagga tgctttaaat tcctacataa attcttccgg tttcgttaat |
| 7501 | tcaggcaaaa agcacattgt cgaatctcca caacttagtt ctagaggtgg tttggactca |
| 7561 | gctacctata tcgccgctct aatcacccac gatattggtg acgatgacac ctacactcca |
| 7621 | ttcaatgtcg acaacagcta tgtcttaaac agtttatatt acttattggt tgataacaag |
| 7681 | aatcgttata aaatcaacgg aaactacaag gctggtgctg ctgttggtag atatcctgaa |
| 7741 | gatgtttaca atggtgtcgg aacttctgaa ggtaatccat ggcaattggc cactgcctac |
| 7801 | gctggtcaaa ctttttatac attagcttac aactccttga agaacaagaa aaatttagta |
| 7861 | attgaaaaat tgaactatga cttgtacaac tctttcatag ctgatctatc gaagatcgat |
| 7921 | agttcctatg caagtaagga ctctttaaca cttacttacg gttccgacaa ttacaaaaac |
| 7981 | gttatcaaat ccttgctaca atttggtgat tcctttttaa aggttttgtt ggatcatatt |
| 8041 | gatgataatg gtcaattaac tgaagaaatt aacagataca ctggttttca agctggcgcc |
| 8101 | gtatcattga catggtcctc cggttctttg ttgtctgcta atagggcaag aaacaaatta |
| 8161 | atcgagctat tataaattga attgaattga aatcgataga tcaatttttt tcttttctct |
| 8221 | ttccccatcc tttacgctaa aataatagtt tattttattt tttgaatatt ttttatttat |
| 8281 | atacgtatat atagactatt atttatcttt taatgattat taagattttt attaaaaaaa |
| 8341 | aattcgctcc tcttttaatg cctttatcca gttttttttt cccattcgat atttctatgt |
| 8401 | tcgggttcag cgtattttaa gtttaataac tcgaaaattc tgcgttcgtt aaagctttcg |
| 8461 | agaaggatat tatttcgaaa taaaccgtgt tgtgtaagct tgaagccttt ttgcgctgcc |
| 8521 | aatattctta tccatctatt gtactcttta gatccagtat agtgtattct tcctgctcca |
| 8581 | agttcatccc acttgcaaca aaactttcga ttagcacgca cacacatcac atagactgcg |
| 8641 | tcataaaaat acactacgga aaaaccataa agagcaaagc gatacctact tggaaggaaa |
| 8701 | aggagcacgc ttgtaagggg gatgggggct aagaagtcat tcactttctt ttcccttcgc |
| 8761 | ggtccggacc cgggacccct cctctccccg cacaatttct tcctttcata tcttcctttt |
| 8821 | attcctatcc cgttgaagca accgcactat gactaaatgg tgctggacat ctccatggct |
| 8881 | gtgacttgtg tgtatctcac agtggtaacg gcaccgtggc tcggaaacgg ttccttcgtg |
| 8941 | acaattctag aacaggggct acagtctcga taatagaata ataagcgcat ttttgttagc |
| 9001 | gccgccgcgg cgcccgtttc ccaataggga ggcgcagttt atcggcggag ctttacttct |
| 9061 | tcctatttgg gtaagcccct ttctgttttc ggccagtggt tgctgcaggc tgcgccggag |
| 9121 | aacatagtga taagggatgt aactttcgat gagagaatta gcaagcggaa aaaaaactat |
| 9181 | ggctagctgg gagttgtttt tcaatcatat aaaagggaga aattgttgct cactatgtga |
| 9241 | cagtttctgg gacgtcttaa cttttattgc agaggactat caaatcatac agatattgtc |
| 9301 | aaaaaaaaaa aaaaagacta ataataaaaa atgatcagat tgactgtctt cttaaccgct |
| 9361 | gttttcgcag ctgtcgcatc ttgtgttccc gttgagcttg acaagagagc cccacaattg |
| 9421 | tcccccaggg ctacttctct agattcctgg ttatccagcg aaactacttt ttctttgaac |
| 9481 | ggtattctcg ccaacatcgg ttcttctggt gcttactcta agtctgctgc ctctggtgcc |
| 9541 | gtcatcgctt ccccttctac tagcaacccc gattactatt atacctggac cagagacgca |
| 9601 | gcgttaactt tgaaagcctt agttgatatt ttccgtaatg gcaatttggg tctacaaacc |
| 9661 | gttatcgaac aatatgttaa tgcacaggct aaattgcaaa ctgtctctaa tccttccgga |
| 9721 | ggtttgtccg acggtgcagg tttgggagaa cctaagttca atgttgactt gtctgctttc |
| 9781 | actggtgctt ggggtagacc acaaagagat ggcccggctc tacgggctat agcactaatc |
| 9841 | gatttcggca attggctgat agataacgga tataaatctt acgcggtgaa caacgtttgg |
| 9901 | ccaatcgtaa ggaacgattt ggcctatgtt gcccagtact ggtcacagtc cggcttcgac |
| 9961 | ctatgggaag aagtgaattc tatgtctttc tttacagttg ctaaccaaca tcgttcatta |
| 10021 | gtcgaaggat cagctttcgc atctcgtgtc ggtgccagct gttctggttg tgactctcaa |
| 10081 | gctcctcaga ttttgtgtta catgcaatct ttttggactg ggagttatat taatgccaat |
| 10141 | acgggtggtg gtagatccgg taaagattct aacactattt tagcctcgat acatactttt |
| 10201 | gatcctgctg cttcttgtga tgacgttacc ttccaaccat gctcaagtag agctttggct |
| 10261 | aaccacaagg tctataccga ttctttcaga tccgtttacg cgttaaactc cggtatagcc |
| 10321 | caaggtaagg ccgtttctgt aggtcgttac ccagaagata gttactacgg tggcaaccca |
| 10381 | tggtttttat caaacttagc agctgctgag caactttatg atgctatcta ccaatggaaa |
| 10441 | aagattggtt ccatcactat cacctcgacc tcgcttgcat ttttcaagga tgtttatccg |
| 10501 | tctgccgcta ccggtaccta tgcttctggg tccacaacct ttaatgctat tatttctgca |
| 10561 | gtaaagacat atgctgacgg ctatgtcagt attgttcaat cccactccta tgcgaatggt |
| 10621 | tcgttgtcag aacaattcga cagaaccact ggtttgtcca tcagtgctcg cgatttaaca |
| 10681 | tggtcttatg cggcgctgtt gactgcaaat gacagaagaa atggcgttgt ccctccatcg |
| 10741 | tggggcgcaa gttccgctaa ttcgatacct ggttcatgca gcatgggttc tgccacaggt |
| 10801 | tcctacgcta ctccatctgt tggttcatgg ccagcaacac ttacttcagg tacagctgca |
| 10861 | ccttccagta catcaactac taccaaggct ccaactacca ccacggccac cacaacaact |
| 10921 | tccgccggtt cctgtactac accaaccgca gtggctgtta ctttcgatga aattgctacg |
| 10981 | acgacatttg gtgaaaacgt ctacttggta ggaagcatta gccaattagg taactggaat |
| 11041 | acagccaacg gtatcccact gtctgcttca aagtacacct cttcaaatcc attatggtac |
| 11101 | gccactgtga acttgcccgc tggcactact tttcaataca aatattttag aaaggaatct |
| 11161 | gatggttcca tcaaatggga gtcagaccca aacagatctt acactgttcc agccaaatgt |
| 11221 | ggtactacta cagccacaga aaatgatact tggagataaa tttaaatgta gatacgttgt |
| 11281 | tgacacttct aaataagcga atttcttatg atttatgatt tttattatta aataagttat |
| 11341 | aaaaaaaata agtgtataca aattttaaag tgactcttag gttttaaaac gaaaattctt |
| 11401 | attcttgagt aactctttcc tgtaggtcag gttgctttct caggtatagc atgaggtcgc |
| 11461 | tcgtttaaac gaatttcgtt gtcacgttgt tttggtaagt tccttcgctt tctcgtaaaa |
| 11521 | ataagtaaaa atccggggaa actattattt gcggttcgaa ataaaagcat tataatttcc |
| 11581 | ttccttggca catttcttgg ccacggatga cctaaaacat tgccaaataa aaaggggtaa |
| 11641 | gagaactt |

SEQ ID NO: 8. HMHG genomic insertion sequence at NLS7

| | |
|---|---|
| FEATURES | Location/Qualifiers |
| misc_feature | 1..500/“UPS_NLS7″ |
| misc_feature | 509..698/″Terminator CYC1″ |
| promoter | 709..1435/Promoter HOR7″ |
| signal_peptide | 1436..1513/″GLM-Signal Peptide″ |
| CDS | 1514..4138/“MALPS21″ |
| terminator | 4139..4566/“Terminator PGK1″ |
| promoter | 4567..5293/″Promoter HOR7″ |
| signal_peptide | 5294..5371/″GLM-Signal peptide″ |
| CDS | 5372..6841/″Glm″ |
| terminator | 6850..7044/″Terminator ADH1″ |
| misc_feature | 7053..7552/“DWS_NLS7″ |

| ORIGIN | |
|---|---|
| 1 | ccattttgag cgagagaacc catttttcta tacaaatttc actagagcac ggccgttaca |
| 61 | tttagtaata gccaataagg gttttttatc gattagtgtt ccctgcgctc cttaacatca |
| 121 | tacaaccgag tccttgacat ggaaatagta ggcaagtaaa ccaaagtcct ttcttcaaaa |
| 181 | gtagaaaact tgagcactta tttcctgcgc atgtcatatg ttaattttcc ttaactgcgc |
| 241 | tgaatacgtc ctgtcaattc aaatatatca cgttttgagc agccctaaag aagaaaacct |
| 301 | caacagcagt attactatta caatcaaaca actttagtgc cgcgtgatac cgggggttga |
| 361 | agtgggtgca ttgagccgta ttcttcttcc ccgtaagaaa gttatgtatc ctttttactg |
| 421 | cgttgtaata gcttctgaaa acctaaaaaa tgaacgctat gtagctcata tccgtttcgc |
| 481 | ataagtaaga ataactactt gtttaaacct tcgagcgtcc caaaaccttc tcaagcaagg |
| 541 | ttttcagtat aatgttacat gcgtacacgc gtttgtacag aaaaaaaaga aaaatttgaa |
| 601 | atataaataa cgttcttaat actaacataa ctataaaaaa ataaataggg acctagactt |
| 661 | caggttgtct aactccttcc ttttcggtta gagcggatat ttcgaaatct ttcgattagc |
| 721 | acgcacacac atcacataga ctgcgtcata aaaatacact acggaaaaac cataaagagc |
| 781 | aaagcgatac ctacttggaa ggaaaaggag cacgcttgta agggggatgg gggctaagaa |
| 841 | gtcattcact ttcttttccc ttcgcggtcc ggacccggga cccctcctct ccccgcacaa |
| 901 | tttcttcctt tcatatcttc cttttattcc tatcccgttg aagcaaccgc actatgacta |
| 961 | aatggtgctg gacatctcca tggctgtgac ttgtgtgtat ctcacagtgg taacggcacc |
| 1021 | gtggctcgga aacggttcct tcgtgacaat tctagaacag gggctacagt ctcgataata |
| 1081 | gaataataag cgcatttttg ttagcgccgc cgcggcgccc gtttcccaat agggaggcgc |
| 1141 | agtttatcgg cggagcttta cttcttccta tttgggtaag cccctttctg ttttcggcca |
| 1201 | gtggttgctg caggctgcgc cggagaacat agtgataagg gatgtaactt tcgatgagag |
| 1261 | aattagcaag cggaaaaaaa actatggcta gctgggagtt gtttttcaat catataaaag |
| 1321 | ggagaaattg ttgctcacta tgtgacagtt tctgggacgt cttaactttt attgcagagg |
| 1381 | actatcaaat catacagata ttgtcaaaaa aaaaaaaaaa gactaataat aaaaaatgat |
| 1441 | cagattgact gtcttcttaa ccgctgtttt cgcagctgtc gcatcttgtg ttcccgttga |
| 1501 | gcttgacaag agagattcat acaccacctc aacagacgat tcgtctaatg acactgccga |
| 1561 | cagtgtctct gatggtgtga ttttacacgc ttggtgttgg tctttcaaca caatcaagaa |
| 1621 | caatttgaag caaattcacg atgcaggtta cactgccgtt caaacctccc ctgtcaatga |
| 1681 | agtcaaagtt ggtaattctg ctagtaagtc tttgaacaac tggtactggt tataccaacc |
| 1741 | aacaaagtac tcgattggta actattactt aggtaccgaa gctgaattca agtccatgtg |
| 1801 | tgcagctgcc aaggagtaca acatcagaat tattgttgat gctaccttga atgacaccac |
| 1861 | aagtgactac tcagctattt cggatgaaat caaatccatt agtaattgga ctcatggcaa |
| 1921 | tacacagata tccaactggt cagacaggga ggatgtcacc caaaactctc tccttggttt |
| 1981 | gtatgattgg aacactcaaa attcccaagt ccaaacatac ctaaagaact acttggaacg |
| 2041 | tctaatatca gatggggcaa gcggttttcg ttacgatgca gccaaacata tcgaattgcc |
| 2101 | atcacaatac gacggttcat atggttccaa tttttggcca aatatcactg acaatggtag |
| 2161 | tgaattccaa tatggcgaag ttttgcaaga ttctatttcc aaagaatccg attacgctaa |
| 2221 | ttacatgtca gtaacagcct ctaattatgg taatactatt agaaatgccc tgaaaaacag |
| 2281 | agatttcact gctagcacat tacaaaattt caatatttct gtccccgcta gcaagttggt |
| 2341 | tacttgggtt gaatctcatg acaactatgc aaacgatgac caagtttcta cctggatgaa |
| 2401 | tagttccgat attaaactag gttgggccgt agtggcctca agatctggaa gtgttccatt |
| 2461 | atttttcgac agaccagttg acggtggtaa tggtacccgt tttcctggat ctagcgaaat |
| 2521 | tggtgacgcc ggttcttcgc tttattatga caaggctgtt gtggcggtta acaagttcca |
| 2581 | caacgccatg gctggtcaat ctgaatacat ttcaaaccca aacggtaaca ccaaaatttt |
| 2641 | tgaaaacgaa agaggttcta agggtgtcgt tttcgctaat gcttcggatg gcagctattc |
| 2701 | tctatctgtt aagacatctc ttgctgacgg tacctacgaa aataaggccg gaagtgacga |
| 2761 | gttcactgtt aaaaacggtt atttgacagg tactatccaa ggtagagaag tagtcgtatt |
| 2821 | atatggcgat ccaacttcaa gctcgtcctc gtctaccact actgaaacta agaaggtgta |
| 2881 | ttttgaaaaa ccatcctcct ggggttccac agtctatgcc tatgtctaca acaaaaacac |
| 2941 | taataaggct ataaccagcg catggccagg taaagagatg actgctttag gtaatgatga |
| 3001 | gtataaatta gacctggata cagatgaaga tgattccgac ttggcagtaa ttttcaccga |
| 3061 | tgggaccaac caaactcctg cagccaacaa ggctgggttc accttcacag cagacgcgac |
| 3121 | gtacgatcag aacggtgttg ttaagacctc tgactcatct tcgtcgtcct ccactaccac |
| 3181 | cgaaacaaaa aaagtgtatt ttgaaaagcc ttcatcttgg gggtccactg tctacgccta |
| 3241 | cgtttataat aaaaacacga acaaagctat caccagtgct tggcccggta aggaaatgac |
| 3301 | cgctcttgga aatgacgaat ataaattgga tttggatact gatgaagatg atagtgatct |
| 3361 | agctgttatc tttactgatg gtacaaacca aacgccggca gctaacaagg caggtttcac |
| 3421 | ttttaccgct gatgccactt atgatcaaaa cggtgtggtt aagacatctg acagttcttc |
| 3481 | atcatcttcc agtacaacta cggaaactaa gaaagtttac ttcgaaaagc catcttcgtg |
| 3541 | gggctctacg gtttacgctt atgtttataa caagaataca aataaagcaa ttacttccgc |
| 3601 | ttggcctggt aaggaaatga ctgcgttagg caacgacgaa tacaagttag atttagatac |
| 3661 | cgatgaagat gatagtgatt tggctgtgat cttcactgat ggaaccaacc agactccagc |
| 3721 | tgctaacaaa gcaggcttta cctttactgc tgatgccact tatgaccaga atggtgttgt |
| 3781 | caagacctcc gatagctcct cttcctcgtc aactactaca gaaacgaaga aggtttactt |
| 3841 | tgagaagcca agtagttggg gttctacagt ttatgcttac gtatacaata aaaatactaa |
| 3901 | taaagcgatc actagcgcct ggccaggtaa agaaatgaca gctttgggca atgacgaata |
| 3961 | caaattggac cttgacactg acgaggacga ctccgatttg gctgttatat ttaccgatgg |
| 4021 | tactaatcaa acgcctgctg caaataaagc tggtttcaca tttaccgccg atgctactta |
| 4081 | cgatcagaac ggtgtcgtca aaacatctga ttcttcgtcc acctcttcta catcataaat |
| 4141 | tgaattgaat tgaaatcgat agatcaattt ttttcttttc tctttcccca tcctttacgc |
| 4201 | taaaataata gtttatttta ttttttgaat attttttatt tatatacgta tatatagact |
| 4261 | attatttatc ttttaatgat tattaagatt tttattaaaa aaaaattcgc tcctctttta |
| 4321 | atgcctttat ccagtttttt tttcccattc gatatttcta tgttcgggtt cagcgtattt |
| 4381 | taagtttaat aactcgaaaa ttctgcgttc gttaaagctt tcgagaagga tattatttcg |
| 4441 | aaataaaccg tgttgtgtaa gcttgaagcc tttttgcgct gccaatattc ttatccatct |
| 4501 | attgtactct ttagatccag tatagtgtat tcttcctgct ccaagttcat cccacttgca |
| 4561 | acaaaacttt cgattagcac gcacacacat cacatagact gcgtcataaa aatacactac |
| 4621 | ggaaaaacca taaagagcaa agcgatacct acttggaagg aaaaggagca cgcttgtaag |
| 4681 | ggggatgggg gctaagaagt cattcacttt cttttccctt cgcggtccgg acccgggacc |
| 4741 | cctcctctcc ccgcacaatt tcttcctttc atatcttcct tttattccta tcccgttgaa |
| 4801 | gcaaccgcac tatgactaaa tggtgctgga catctccatg gctgtgactt gtgtgtatct |
| 4861 | cacagtggta acggcaccgt ggctcggaaa cggttccttc gtgacaattc tagaacaggg |
| 4921 | gctacagtct cgataataga ataataagcg catttttgtt agcgccgccg cggcgcccgt |
| 4981 | ttcccaatag ggaggcgcag tttatcggcg gagctttact tcttcctatt tgggtaagcc |
| 5041 | cctttctgtt ttcggccagt ggttgctgca ggctgcgccg gagaacatag tgataaggga |
| 5101 | tgtaactttc gatgagagaa ttagcaagcg gaaaaaaaac tatggctagc tgggagttgt |
| 5161 | ttttcaatca tataaaaggg agaaattgtt gctcactatg tgacagtttc tgggacgtct |
| 5221 | taacttttat tgcagaggac tatcaaatca tacagatatt gtcaaaaaaa aaaaaaaaga |
| 5281 | ctaataataa aaaatgatca gattgactgt cttcttaacc gctgttttcg cagctgtcgc |
| 5341 | atcttgtgtt cccgttgagc ttgacaagag aaatacaggt catttccaag cctactctgg |
| 5401 | ttacacagtt gctcgttcca acttcaccca atggattcac gaacaacctg ccgtgtcatg |
| 5461 | gtattatttg cttcagaata ttgactaccc agaaggccag ttcaaatcgg ccaagcctgg |
| 5521 | tgttgttgtg gccagcccat ctacttcaga gccagattac ttttaccaat ggactagaga |
| 5581 | tactgcaatt actttcttga gtttgattgc tgaagttgaa gaccattctt tttcaaacac |
| 5641 | tactttggct aaggtcgttg aatactacat ttcaaataca tacaccttac aaagagtatc |
| 5701 | gaacccatca ggtaactttg acagcccaaa ccatgatggt ttaggtgaac caaagtttaa |
| 5761 | tgtggatgat accgcatata ctgcttcttg gggtcgtcct caaaatgacg gtccagcttt |
| 5821 | gagagcttat gctatttcta ggtatctgaa tgccgtcgcc aaacacaaca acggtaagtt |
| 5881 | gctgctcgcg ggccaaaacg gtataccgta ttcttctgcc tctgatatct actggaaaat |
| 5941 | tattaaacct gatttacaac atgtttccac ccattggtct acctccggat ttgatttgtg |
| 6001 | ggaagagaac caaggtactc acttcttcac ggcactagtg cagttgaaag ctctatctta |
| 6061 | tggtattcct ttgtccaaga cttataatga tccagggttt acctcgtggt tggaaaagca |
| 6121 | aaaggatgct ttaaattcct acataaattc ttccggtttc gttaattcag gcaaaaagca |
| 6181 | cattgtcgaa tctccacaac ttagttctag aggtggtttg gactcagcta cctatatcgc |
| 6241 | cgctctaatc acccacgata ttggtgacga tgacacctac actccattca atgtcgacaa |
| 6301 | cagctatgtc ttaaacagtt tatattactt attggttgat aacaagaatc gttataaaat |
| 6361 | caacggaaac tacaaggctg gtgctgctgt tggtagatat cctgaagatg tttacaatgg |
| 6421 | tgtcggaact tctgaaggta atccatggca attggccact gcctacgctg gtcaaacttt |
| 6481 | ttatacatta gcttacaact ccttgaagaa caagaaaaat ttagtaattg aaaaattgaa |
| 6541 | ctatgacttg tacaactctt tcatagctga tctatcgaag atcgatagtt cctatgcaag |
| 6601 | taaggactct ttaacactta cttacggttc cgacaattac aaaaacgtta tcaaatcctt |
| 6661 | gctacaattt ggtgattcct ttttaaaggt tttgttggat catattgatg ataatggtca |
| 6721 | attaactgaa gaaattaaca gatacactgg ttttcaagct ggcgccgtat cattgacatg |
| 6781 | gtcctccggt tctttgttgt ctgctaatag ggcaagaaac aaattaatcg agctattata |
| 6841 | aatttaaatg tagatacgtt gttgacactt ctaaataagc gaatttctta tgatttatga |
| 6901 | tttttattat taaataagtt ataaaaaaaa taagtgtata caaattttaa agtgactctt |
| 6961 | aggttttaaa acgaaaattc ttattcttga gtaactcttt cctgtaggtc aggttgcttt |
| 7021 | ctcaggtata gcatgaggtc gctcgtttaa acaaaaccgc tgcagcaacc cttgttacat |
| 7081 | acagtcggat ccatctgact tactttcctt gcgtctccct gcgcgatctt gttggccatt |
| 7141 | ttccagatcc tctagaattt ttcaagggtc gagccgtagg aggattctct cagaaggcaa |
| 7201 | aaacgcatcg aaagcgtgct ttgtaagaat atttggtatg gctaaagtaa gcaaagccat |
| 7261 | atcccgatcc cgatcccgac tcttattccg atcccttctg ccacatcctg catgtttatt |
| 7321 | cgaataccga attagctcat cttcgttatt ttcatcatcc ctttctgcta tagcaaggac |
| 7381 | aagttttttt ctagcatctc atcgaaaact ttcctctccc taattggcca aagttttcat |
| 7441 | attcatcatc agttagaaag tataatatca atcccttacc tcattacaag ttgtatcaca |
| 7501 | ctaaaaaaat catatataag tctgtgagag tcttcaatta tttagcgtaa ca |

### DETAILED DESCRIPTION OF THE INVENTION

For the purposes of promoting an understanding of the principles of the novel technology, reference will now be made to the preferred embodiments thereof, and special language will be used to describe the same. It will nevertheless be understood that no limitation of the scope of the novel technology is thereby intended, such alterations, modifications, and further applications of the principles of the novel technology being contemplated as would normally occur to one skilled in the art to which the novel technology relates.

As used herein, unless specified otherwise, the term ‘about’ means plus or minus 20 percent, for example, about 1.0 encompasses the range 0.8 to 1.2.

Unless specifically referred to otherwise, genes are referred to using the nomenclature suggested by Demerec et al., A proposal for a uniform nomenclature in bacterial genetics. J. GEN. MICROBIOL (1968) 50, 1-14.

A “vector” is any nucleic acid molecule for the cloning of and/or transfer of a nucleic acid into a cell. A vector may be a replicon to which another nucleotide sequence may be attached to allow for replication of the attached nucleotide sequence.

A “recombinant” vector refers to a viral or non-viral vector that comprises one or more exogenous nucleotide sequences (i.e., trans genes), e.g., two, three, four, five or more exogenous nucleotide sequences. An “expression” vector refers to a viral or non-viral vector that is designed to express a product encoded by an exogenous nucleotide sequence inserted into the vector.

The term “exogenous” with respect to a polynucleotide means a polynucleotide that is not native to the cell in which it is located or, alternatively, a polynucleotide which is normally found in the cell but is in a different location or is expressing different copy number than normal (e.g., in a vector or in a different location in the genome).

The term “recombinant organism” refers to any organism including, but is not limited to, a strain or a part of yeast whose genetic material has been altered using genetic engineering techniques. In any one of the embodiments disclosed herein, the polynucleotide can be inserted into a cell of an organism including, but is not limited to, a strain or a part of yeast by genetic engineering (e.g., insertion of an expression vector).

The term “express” or “expression” of a polynucleotide coding sequence means that the sequence is transcribed, and optionally, translated. Typically, according to the present invention, expression of a coding sequence of the invention will result in production of the polypeptide of the invention. The entire expressed polypeptide or fragment can also function in intact cells without purification.

As used herein, the terms “protein” and “polypeptide” can be interchangeably used and can encompass both peptides and proteins, unless specifically indicated otherwise.

For those skilled in the art, protein sequence similarity is calculated by alignment of two protein sequences. Commonly used pairwise alignment tools include COBALT (Papadopoulos and Agarwala, 2007), EMBOSS Needle (Needleman and Wunsch, 1970) and EMBOSS Stretcher (Myers and Miller, 1988). The percentage of identity represents the total fraction of amino acids that are identical along the length of each protein. Similarity is calculated based on the percentage of amino acids with similar character over the reported aligned region. Amino acids are considered similar if they share common chemical properties that impart similar qualities to the structure and activity of the entire protein.

The construction of F20 strain was achieved by two consecutive integrations of selected glucoamylases and maltogenic alpha-amylase enzymes cassettes at neutral landing sites (NLS) of 3 and 7 respectively in the parent strain, ER-19-11-4, which we have previously described in U.S. patent application Ser. No. 17/261,454, as discussed above . . .

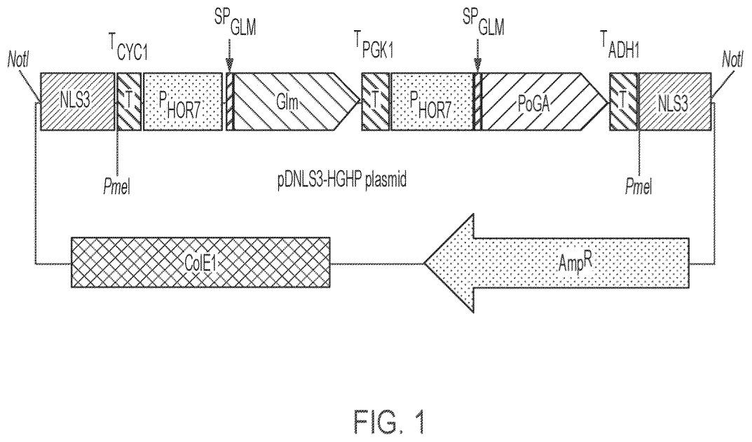

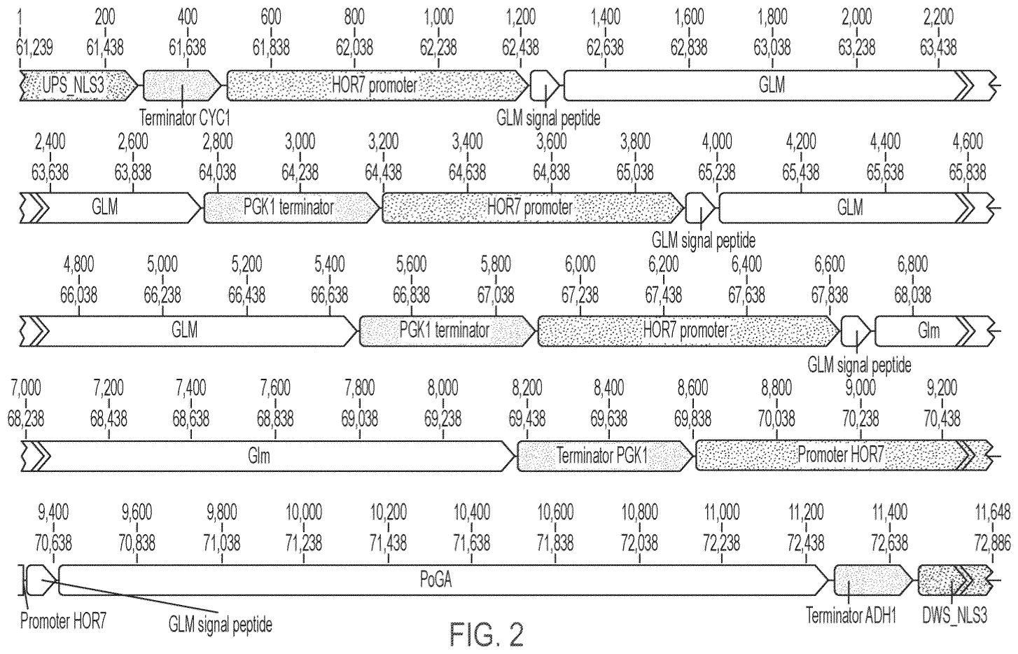

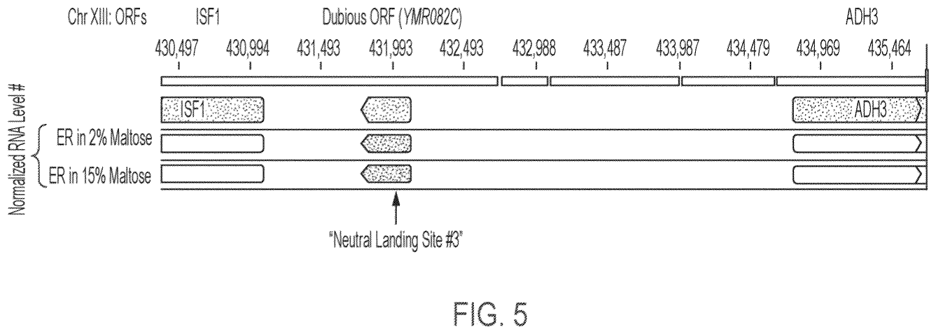

The first integration cassette includes glucoamylases, namely GLM of Saccharomycopsis fibuligera and PoGA of Penicillium oxalicum under the HOR7 promoter. Both the glucoamylases gene sequences used in the construction of HGHP cassette were codon optimized for S. cerevisiae and synthesized as gblock DNA fragments (IDT, Coralville, IA, USA). The HOR7 promoter, CYC1, PGKI and ADHI terminator sequences were PCR amplified from the genomic DNA Ethanol Red strain using Q5 PCR reaction mixture (New England Biolabs). The overlapping PCR fragments were gel purified and then cloned into Pmel linearized target vector backbone of pDNLS3 (FIG. 1) using HiFi DNA assembly kit as recommended in the manufacturer's protocol (New England Biolabs). The correct vector assembly with desired genetic components was verified by PCR and sequencing. The DNA of verified HGHP gene cassette was digested with Notl restriction enzyme and gel purified as linear DNA fragments for integration into the designated Neutral Landing Site 3 of selected S. cerevisiae strains using CRISPR technology. The linear DNA fragment of HGHP cassette and plasmid DNA expressing both the nuclease and NLS3-targeting gRNA were transformed into S. cerevisiae according to a previously published protocol (Gietz et al., Yeast transformation by the LiAc/SS Carrier DNA/PEG method, METHODS MOL BIOL 2006, 313:107-120). The transformed cells were plated on selective YPD media plates supplemented with 50 μg/ml of G418 antibiotic. Plates were incubated at 30° C. for 2-3 days, until colonies became visible. Upon appearance of visible colonies on YPD plates, integration of HGHP gene cassette at the NLS3 site was confirmed by starch hydrolysis assay and subsequently integration of HGHP DNA fragment in selected positive clones were confirmed via direct colony PCR prior to long term storage in 15% glycerol at −80° C. The resulting strain is known to us as F15-NLS3-HGHP-101. Neutral Landing Site (NLS3) was selected as the site of HGHP cassette integration for several regions. First, to avoid disrupting any important genetic elements; a spot-on chromosome XIII overlapping the dubious open reading frame YMR082C but sufficiently distant from other annotated genes was chosen. Genome-wide RNA expressions were measured in Fermentis Ethanol Red fermenting either maltose or glucose at both high (15%) and low (2%) concentrations. Under all conditions tested the genes neighboring NLS3 are expressed at moderate levels indicating that this is a region amenable to Pol II transcription under a wide variety of conditions (FIG. 5). Together the analyses disclosed herein indicate the region overlapping YMR082C provides a suitable and stable platform where superior genetic traits can be engineered in Ethanol Red and their derivative strains.

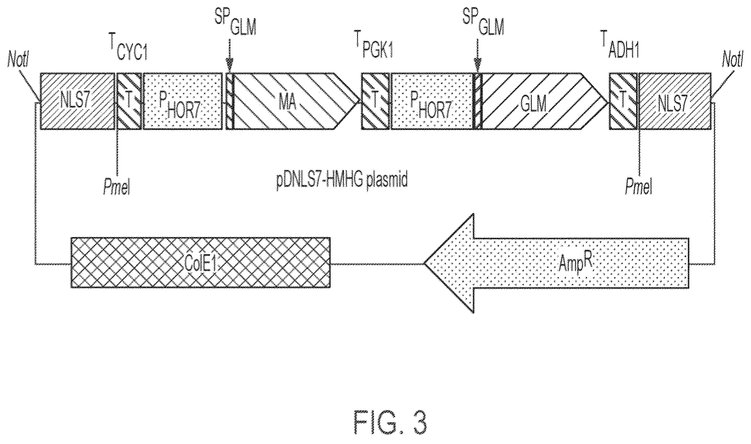

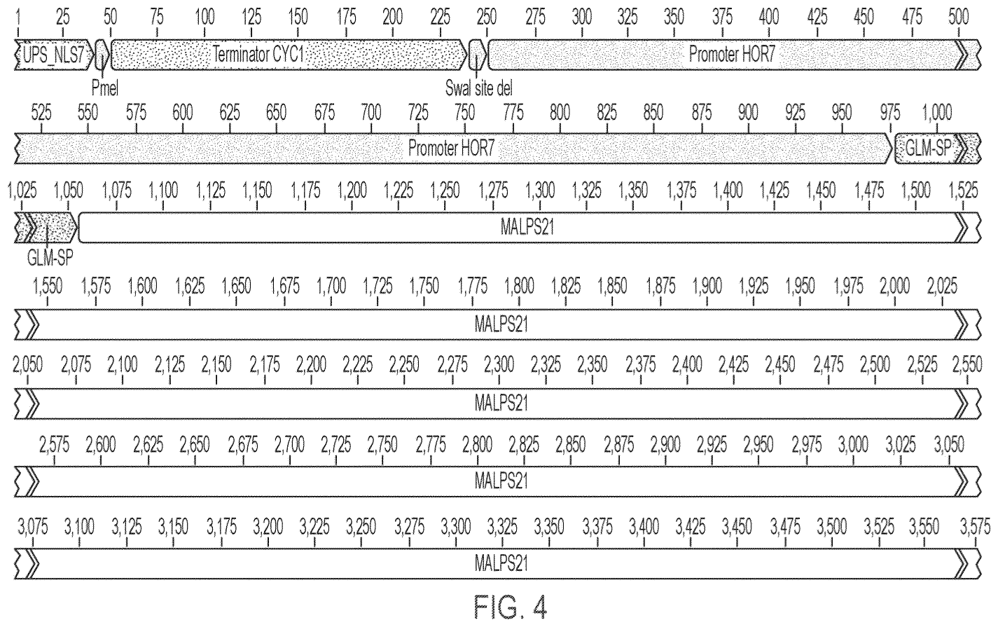

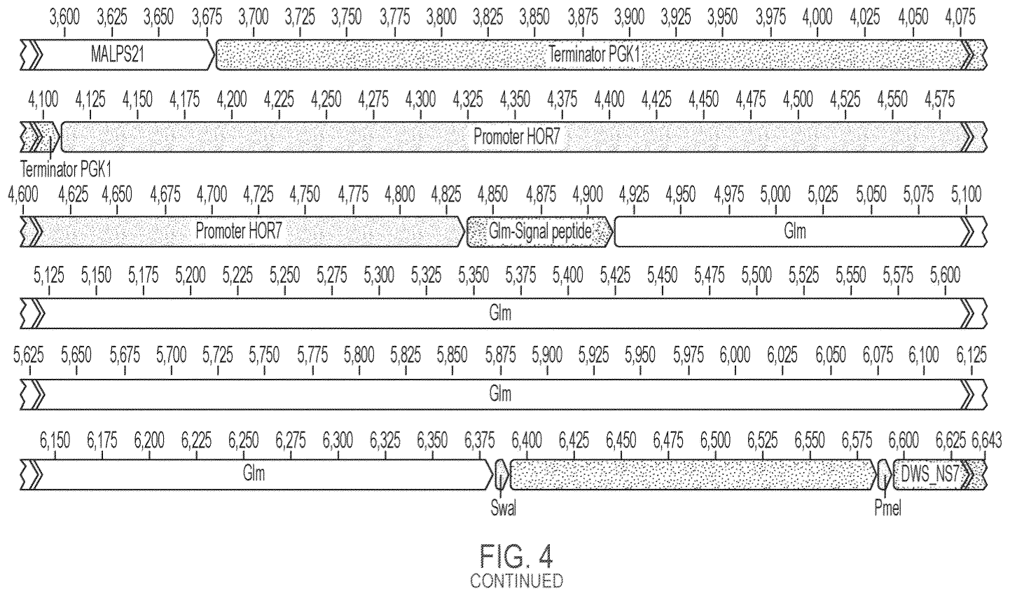

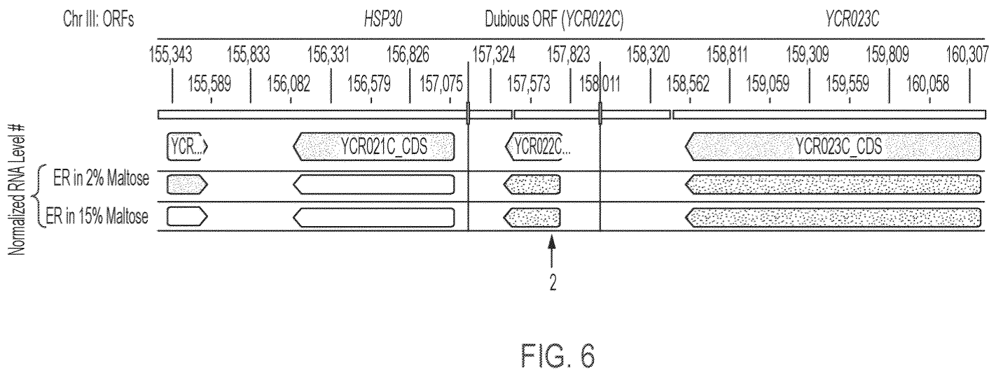

The second integration cassette consists of two glucoamylases namely a maltogenic alpha amylase of Lactobacillus plantarum S21 and GLM of Saccharomycopsis fibuligera under HOR7 promoter. Both amylase gene sequences used in the construction of HMHG cassette were codon optimized for S. cerevisiae and synthesized as gblock DNA fragments (IDT, Coralville, IA, USA). The HOR7 promoter, CYC1, PGK1 and ADH1 terminator sequences were PCR amplified from the genomic DNA Ethanol Red strain using Q5 PCR reaction mixture (New England Biolabs). The overlapping PCR fragments were gel purified and then cloned into Pmel linearized target vector backbone of pDNLS7 (FIG. 3) using HiFi DNA assembly kit as recommended in the manufacturer's protocol (New England Biolabs). The correct vector assembly with desired genetic components was verified by PCR and sequencing. The DNA of verified HMHG gene cassette was digested with Notl restriction enzyme and gel purified as linear DNA fragments for integration into the designated Neutral Landing Site 7 of F15-NLS3-HGHP-101 or other selected S. cerevisiae strains using CRISPR technology. The linear DNA fragment of HMHG cassette and plasmid DNA expressing both the nuclease and NLS7-targeting gRNA were transformed into S. cerevisiae according to a previously published protocol (Gietz et al., Yeast transformation by the LiAc/SS Carrier DNA/PEG method, METHODS MOL BIOL 2006, 313:107-120). The transformed cells were plated on selective YPD media plates supplemented with 50 μg/ml of G418 antibiotic. Plates were incubated at 30° C. for 2-3 days, until colonies became visible. Upon appearance of visible colonies on YPD plates, integration of HMHG gene cassette at the NLS7 site was confirmed by starch hydrolysis assay and subsequently integration of HMHG DNA fragment in selected positive clones were confirmed via direct colony PCR prior to long term storage in 15% glycerol at −80° C. The resulting strain is known to us as F15-10-MG-57 (aka F20). Neutral Landing Site (NLS7) was selected as the site of HMHG cassette integration for several regions. First, to avoid disrupting any important genetic elements; a spot-on chromosome III overlapping the dubious open reading frame YCR022C but sufficiently distant from other annotated genes was chosen. Genome-wide RNA expressions were measured in Fermentis Ethanol Red fermenting either maltose or glucose at both high (15%) and low (2%) concentrations. Under all conditions tested the genes neighboring NLS7 are expressed at moderate levels indicating that this is a region amenable to Pol II transcription under a wide variety of conditions (FIG. 6). Still referring to FIG. 6, 2 denotes Neutral Landing Site #7. Together, the analyses disclosed herein indicate the region overlapping YCR022C provides a suitable and stable platform where superior genetic traits can be engineered in Ethanol Red and their derivative strains.

### EXPERIMENTAL

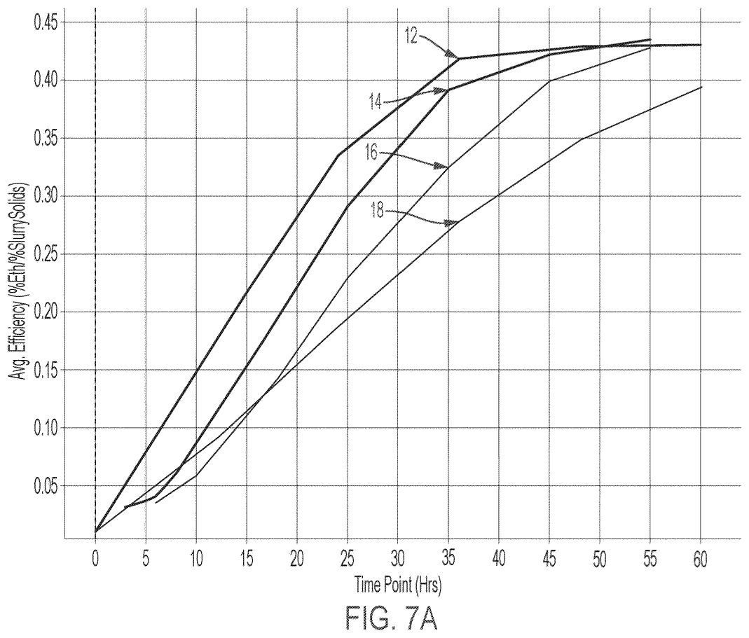

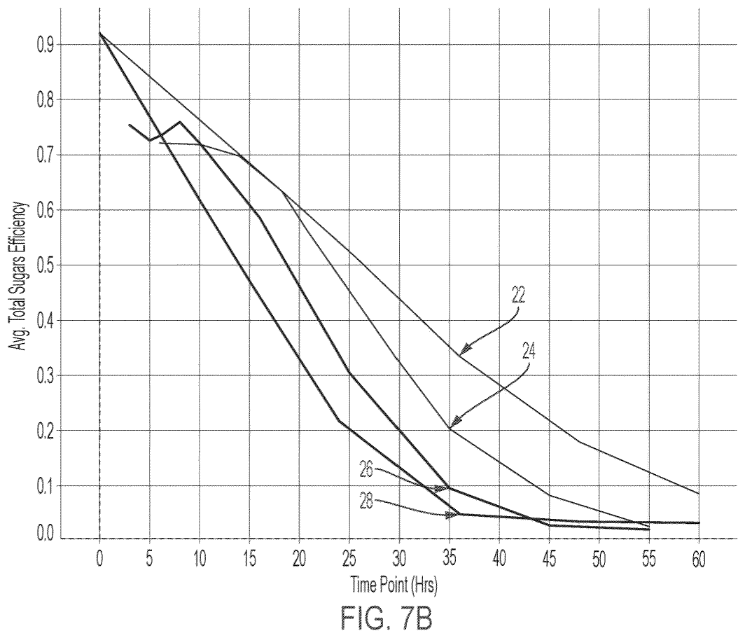

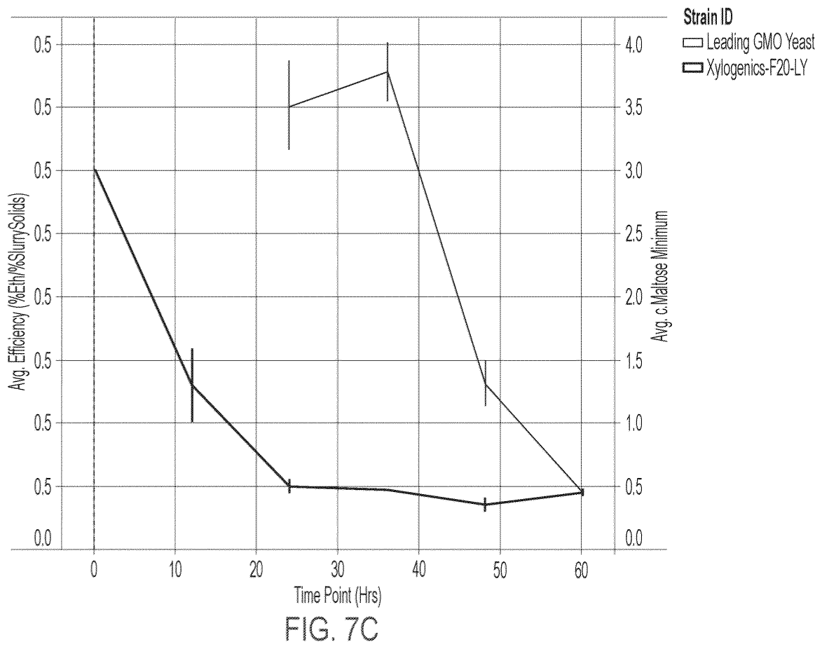

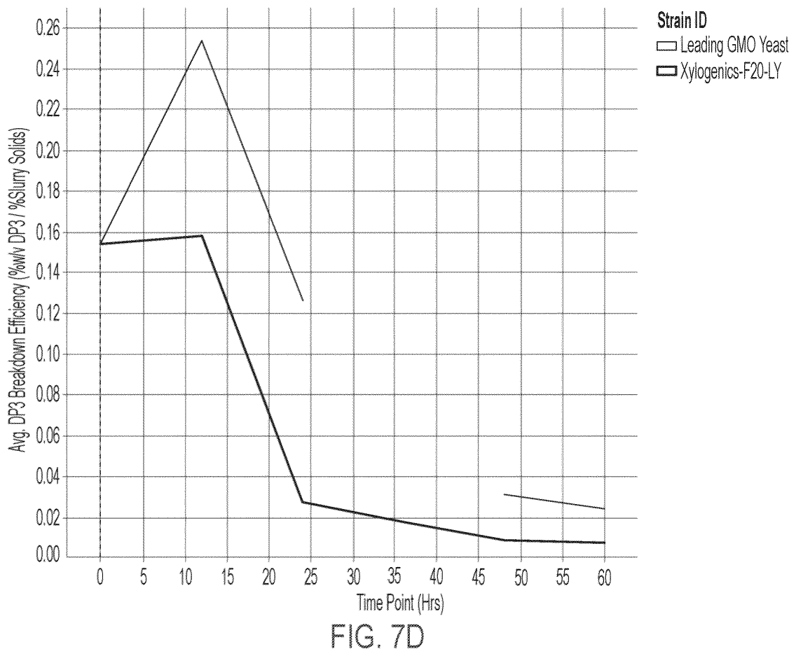

To test the fermentation ability of F20, a liquid corn mash slurry containing 33.25% solids was treated with a 0.02% solution of Ultra F glucoamylase (Novozymes). F20 rapidly broke down the DP4+ sugars to produce 12.84% (w/v) ethanol after 35 hours (FIG. 7A) with only 2.99% (w/v) average total sugars remaining (FIG. 7B). This can be directly compared to Innova Force, the leading industrial strain, which, when introduced to a liquid corn mash slurry containing 33.25% solids and treated with a 0.02% solution of Ultra F glucoamylase, yields 11.05% (w/v) ethanol (FIG. 7A) and 6.76% average total sugars after 35 hours (FIG. 7B). F20 also consumes maltose and DP3 sugars more quickly than Innova Force due to F20's lack of glucose repression (FIGS. 7C and 7D). F20 yeast in a liquid corn mash slurry of 33.25% solids treated with 0.02% Ultra F glucoamylase starts quicker, co-consumes DP3 and maltose sugars more readily, finishes fermentation faster, and produces more ethanol with lower average total sugars than the leading industrial strain (FIGS. 7A-D).

Referring to FIG. 7A: 12 denotes the curve determined using Xylogenics-F20-LY, GA Dosage % (w/w)-0, Ethanol % (w/w)-14.43; 14 denotes the curve determined using Xylogenics-F20-LY, GA Dosage % (w/w)-0.02, Ethanol % (w/w)-12.84; 16 denotes the curve determined using the Leading GMO Yeast, GA Dosage % (w/w)-0.02, Ethanol % (w/w)-11.05; 18 denotes the curve determined using the Leading GMO Yeast, GA Dosage % (w/w)-0.0, Ethanol % (w/w)-9.58. Briefly, Xylogenics F-20, appears to be a true 0, GA, or at least very close to 0, GA, yeast and it exhibits fast fermentation kinetics. Xylogenics F20 Yeast is fast at producing ethanol while maintaining a high starch to ethanol conversion efficiency. This yeast drastically reduced (0.02% w/w) GA doses and gets better as the GS is lowered to 0. F20's accelerated kinetics allows for fermentive versatility and ultimately grate operational freedom.

Referring to FIG. 7B: 22 denotes the curve determined using the Leading GMO Yeast, GA Dosage % (w/w)-0, Average Total Sugars (% w/w)-11.44; 24 denotes the curve determined using the Leading GMO Yeast, GA Dosage % (w/w)-0.2, Average Total Sugars (% w/w)-6.76; 26 denotes the curve determined using the Xylogenics-F20-LY, GA Dosage % (w/w)-0.2, Average Total Sugars (% w/w)-2.99; 28 denotes the curve determined using the Xylogenics-F20-LY, GA Dosage % (w/w)-0, Average Total Sugars (% w/w)-1.54. Briefly, Xylogenics-F20 includes a novel combination of technologies that unexpectedly accelerates starch hydrolysis and its conversion to ethanol.

As a second test, F20 was introduced into a liquid corn mash slurry with 34.49% solids but was not supplemented with exogenous glucoamylase, instead relying on the expression of endogenous glucoamylases and maltogenic alpha amylase. Even without supplemental glucoamylases, F20 rapidly broke down the DP4+ sugars to produce 14.43% (w/v) ethanol after 35 hours (FIG. 7A) with only 1.54% (w/v) average total sugars remaining (FIG. 7B). This can be compared to Innova Force, the leading industrial strain, which, when introduced to a liquid corn mash slurry containing 34.49% solids without supplemental glucoamylase, yields 9.58% (w/v) ethanol (FIG. 7A) and 11.44% average total sugars after 35 hours (FIG. 7B). F20 yeast in a liquid corn mash slurry of 34.49% solids with zero glucoamylase supplementation, starts and finishes fermentation faster and produces more ethanol with lower average total sugars than the leading industrial strain (FIGS. 7A and 7B).

Performance of F20 yeast improves without any supplemental glucoamylase when compared to examples including 0.02% glucoamylase supplementation. F20 produces ethanol more efficiently during the first 40 hours of fermentation (FIG. 7A) and consumes the total pool of corn mash sugars more efficiently for the first 40 hours of fermentation (FIG. 7B) when no glucoamylase is added to the fermentation. The overall ethanol production and total sugar efficiency is very similar at the finish of fermentation, within 0.1, for F20 yeast with or without glucoamylase. This is in contrast to Innova Force which is much less efficient throughout a fermentation when no glucoamylase is added and fermentations are incomplete in the absence of exogenous glucoamylase (FIGS. 7A and 7B).

We claim:

1. A recombinant yeast strain, comprising:
a strain of S. cerevisiae;
an exogenous MALI gene cluster, wherein the strain of S. cerevisiae expresses the exogenous MALI gene cluster;
an exogenous MAL2-8c gene, wherein the strain of S. cerevisiae expresses the exogenous MAL2-8c gene; and
an exogenous maltogenic alpha amylase gene from Lactobacillus plantarum S21, wherein the strain of S. cerevisiae expresses the exogenous maltogenic alpha amylase gene from Lactobacillus plantarum S21.

2. The recombinant yeast strain according to claim 1, wherein the exogenous maltogenic alpha amylase from Lactobacillus plantarum S21 gene is overexpressed.

3. The recombinant yeast strain according to any one of claims 1 and 2, further comprising an exogenous glucoamylase gene from Saccharomycopsis fibuligera.

4. The recombinant yeast strain according to claim 3, wherein the exogenous glucoamylase gene from Saccharomycopsis fibuligera gene is overexpressed.

5. The recombinant yeast strain according to any one of claims 3 and 4, wherein the exogenous glucoamylase gene from Saccharomycopsis fibuligera is present in more than one copy per cell.

6. The recombinant yeast strain according to any one of claims 3-5, wherein the exogenous glucoamylase gene from Saccharomycopsis fibuligera is integrated into the genome of the strain of S. cerevisiae.

7. The recombinant yeast strain according to claim 6, wherein the exogenous glucoamylase gene from Saccharomycopsis fibuligera is integrated into the genome at different positions on more than one chromosome.

8. The recombinant yeast strain according to any one of claims 3-7, wherein the exogenous glucoamylase gene from Saccharomycopsis fibuligera is inserted into the genome of the strain of S. cerevisiae within a region encoding the Dubious Open Reading Frame YCR022c.

9. The recombinant yeast strain according to any one of claims 3-8, wherein the exogenous glucoamylase gene from Saccharomycopsis fibuligera is inserted into the genome of the strain of S. cerevisiae within a region encoding the Dubious Open Reading Frame YMR082c.

10. The recombinant yeast strain according to any one of claims 8 and 9, wherein the exogenous glucoamylase gene from Saccharomycopsis fibuligera is inserted into two places of the genome of the strain S. cerevisiae, a first region encoding the Dubious Open Reading Frame YCR022c and a second region encoding the Dubious Open Reading Frame YMR082c.

11. The recombinant yeast strain according to any one of claims 3-10, wherein the exogenous glucoamylase gene from Saccharomycopsis fibuligera comprises a sequence having at least 80% homology to SEQ ID NO: 3 and the exogenous maltogenic alpha amylase gene from Lactobacillus plantarum S21 comprises a sequence having at least 80% homology to SEQ ID NO: 1.

12. The recombinant yeast strain according to any one of claims 3-11, wherein the exogenous glucoamylase gene from Saccaromycopsis fibuligera comprises a sequence having at least 85% homology to SEQ ID NO: 3 and the exogenous maltogenic alpha amylase gene from Lactobacillus plantarum S21 comprises a sequence having at least 85% homology to SEQ ID NO: 1.

13. The recombinant yeast strain according to any one of claims 3-12, wherein the exogenous glucoamylase gene from Saccaromycopsis fibuligera comprises a sequence having at least 90% homology to SEQ ID NO: 3 and the exogenous maltogenic alpha amylase gene from Lactobacillus plantarum S21 comprises a sequence having at least 90% homology to SEQ ID NO: 1.

14. The recombinant yeast strain according to any one of claims 3-13, wherein the exogenous glucoamylase gene from Saccaromycopsis fibuligera comprises a sequence having at least 95% homology to SEQ ID NO: 3 and the exogenous maltogenic alpha amylase gene from Lactobacillus plantarum S21 comprises a sequence having at least 95% homology to SEQ ID NO: 1.

15. The recombinant yeast strain according to any one of claims 3-14, wherein the exogenous glucoamylase gene from Saccaromycopsis fibuligera comprises a sequence having SEQ ID NO: 3 and the exogenous maltogenic alpha amylase gene from Lactobacillus plantarum S21 comprises a sequence having SEQ ID NO: 1.

16. The recombinant yeast strain according to any one of claims 1-15, further comprising an exogenous glucoamylase gene from Penicillium oxalicum.

17. The recombinant yeast strain according to claim 16, wherein the exogenous glucoamylase gene from Penicillium oxalicum gene is overexpressed.

18. The recombinant yeast strain according to any one of claims 16 and 17, wherein the exogenous glucoamylase gene from Penicillium oxalicum is integrated into the genome of the strain of S. cerevisiae.

19. The recombinant yeast strain according to any one of claims 16-18, wherein the exogenous glucoamylase gene from Penicillium oxalicum is inserted into the genome of the strain of S. cerevisiae within a region encoding the Dubious Open Reading Frame YMR082c.

20. The recombinant yeast strain according to any one of claims 16-19, wherein the exogenous glucoamylase gene from Penicillium oxalicum comprises a sequence having at least 80% homology to SEQ ID NO: 5 and the exogenous maltogenic alpha amylase gene from Lactobacillus plantarum S21 comprises a sequence having at least 80% homology to SEQ ID NO: 1.

21. The recombinant yeast strain according to any one of claims 16-20, wherein the exogenous glucoamylase gene from Penicillium oxalicum comprises a sequence having at least 85% homology to SEQ ID NO: 5 and the exogenous maltogenic alpha amylase gene from Lactobacillus plantarum S21 comprises a sequence having at least 85% homology to SEQ ID NO: 1.

22. The recombinant yeast strain according to any one of claims 16-21, wherein the exogenous glucoamylase gene from Penicillium oxalicum comprises a sequence having at least 90% homology to SEQ ID NO: 5 and the exogenous maltogenic alpha amylase gene from Lactobacillus plantarum S21 comprises a sequence having at least 90% homology to SEQ ID NO: 1.

23. The recombinant yeast strain according to any one of claims 16-22, wherein the exogenous glucoamylase gene from Penicillium oxalicum comprises a sequence having at least 95% homology to SEQ ID NO: 5 and the exogenous maltogenic alpha amylase gene from Lactobacillus plantarum S21 comprises a sequence having at least 95% homology to SEQ ID NO: 1.

24. The recombinant yeast strain according to any one of claims 16-23, wherein the exogenous glucoamylase gene from Penicillium oxalicum comprises a sequence having SEQ ID NO: 5 and the exogenous maltogenic alpha amylase gene from Lactobacillus plantarum S21 comprises a sequence having SEQ ID NO: 1.

25. The recombinant yeast strain according to any one of claims 1-24, wherein the exogenous maltogenic alpha amylase gene from Lactobacillus plantarum S21 is integrated into the genome of the strain of S. cerevisiae.

26. The recombinant yeast strain according to any one of claims 1-25, wherein the exogenous maltogenic alpha amylase gene from Lactobacillus plantarum S21 is inserted into the genome of the strain of S. cerevisiae within a region encoding the Dubious Open Reading Frame YCR022c.

27. The recombinant yeast strain according to any one of claims 1-26, wherein the strain of S. cerevisiae is haploid, diploid, or has a ploidy number greater than two.

28. The recombinant yeast strain according to any one of claims 1-27, wherein the recombinant yeast strain is made using genetic engineering or wherein the recombinant yeast strain is genetically modified.

29. The recombinant yeast strain according to any one of claims 1-28, wherein the recombinant yeast strain is capable of fermenting maltose as well as disaccharides and trisaccharides comprised of glucose while simultaneously improving the efficiency and speed of glucose fermentation and eliminating the requirement for supplemental glucoamylase.

30. A vector, comprising:
a maltogenic alpha amylase gene from Lactobacillus plantarum S21 that comprises a sequence having at least 80%, at least 85%, at least 90%, at least 95%, at least 98%, or 100% percent homology or identity to SEQ ID NO: 1.

31. The vector according to claim 30, further comprising a glucoamylase gene from Saccharomycopsis fibuligera that comprises a sequence having at least 80%, at least 85%, at least 90%, at least 95%, at least 98%, or 100% percent homology or identity to SEQ ID NO: 3.

32. The vector according to claim 31, further comprising a glucoamylase gene from Penicillium oxalicum that comprises a sequence having at least 80%, at least 85%, at least 90%, at least 95%, at least 98%, or 100% homology or identity to SEQ ID NO: 5.

33. The vector according to any one of claims 30-32, wherein the maltogenic alpha amylase gene from Lactobacillus plantarum S21 and/or the glucoamylase gene from Saccharomycopsis fibuligera and/or the glucoamylase gene from Penicillium oxalicum are maintained and expressed in a haploid, diploid, or polyploid of the strain of S. cerevisiae.

34. The vector according to any one of claims 30-33, wherein the vector is expressed in the strain of S. cerevisiae as a single copy or multiple copies.

35. A vector, comprising:
a glucoamylase gene from Penicillium oxalicum that comprises a sequence having at least 80%, at least 85%, at least 90%, at least 95%, at least 98%, or 100% percent homology or identity to SEQ ID NO: 5.

36. The vector according to claim 35, wherein the glucoamylase gene from Penicillium oxalicum is maintained and expressed in a haploid, diploid, or polyploid of a strain of S. cerevisiae.

37. The vector according to claim 36, wherein the vector is expressed in the strain of S. cerevisiae as a single copy or multiple copies.

38. A vector, comprising:
a glucoamylase gene from Saccharomycopsis fibuligera having at least 80%, at least 85%, at least 90%, at least 95%, at least 98%, or at least 100% homology or identity to SEQ ID NO: 3.

39. The vector according to claim 38, wherein the glucoamylase gene from Saccharomycopsis fibuligera is maintained and expressed in a haploid, diploid, or polyploid of a strain of S. cerevisiae.

40. The vector according to claim 39, wherein the vector is expressed in the strain of S. cerevisiae as a single copy or multiple copies.

41. A method of producing a recombinant yeast strain, comprising:
integrating an exogenous maltogenic alpha amylase gene from Lactobacillus plantarum S21 having at least 80% homogeny to SEQ ID NO: 1 and/or an exogenous glucoamylase gene from Saccharomycopsis fibuligera having at least 80% homogeny to SEQ ID NO: 3 and/or an exogenous glucoamylase gene from Penicillium oxalicum having at least 80% homogeny to SEQ ID NO: 5 into the genome of a strain of S. cerevisiae.
